(19) United States
(12) Patent Application Publication
(10) Pub. No.: US 2015/0087671 A1
(43) Pub. Date: Mar. 26, 2015

(54) LOW BURST SUSTAINED RELEASE LIPOPHILIC AND BIOLOGIC AGENT COMPOSITIONS

(71) Applicant: Micell Technologies, Inc., Durham, NC (US)

(72) Inventors: James B. McClain, Raleigh, NC (US); Charles Douglas Taylor, Franklinton, NC (US); Wenda C. Carlyle, Prairie Farm, WI (US)

(21) Appl. No.: 14/401,496

(22) PCT Filed: May 16, 2013
(86) PCT No.: PCT/US13/41466

§ 371 Date: Nov. 14, 2014

Related U.S. Application Data
(60) Provisional application No. 61/648,019, filed on May 16, 2012.

(51) Int. Cl. A61K 47/10; A61L 31/06; A61L 31/16; A61K 31/436; A61K 9/06

(52) U.S. Cl. CPC A61K 47/10; A61K 31/436; A61K 9/06; A61L 31/16; A61L 31/06; A61L 2300/602
USPC 514/291

(57) ABSTRACT

A drug delivery composition including a lipophilic agent or a biologic agent and a polymer wherein the lipophilic agent exhibits sustained release and wherein there is less than 35% agent release within the first hour of elution. A drug delivery composition including a lipophilic agent or a biologic agent and a polymer wherein the elution profile is substantially linear, and wherein there is less than 35% agent release within the first hour of elution. The lipophilic agent may be crystalline and the biologic agent may be in active form.

### CROSS-REFERENCE

This application claims the benefit of U.S. provisional application No. 61/648,019, filed May 16, 2012, which is incorporated by reference in its entirety.

### BACKGROUND OF THE INVENTION

Oral administration of rapamycin suffers from poor bioavailability and once-daily dosing results in fluctuating peak and trough blood levels. Immunosuppressive effectiveness of rapamycin and other limus compounds is dose dependent requiring that trough levels of drug remain in the therapeutic range. Too high blood levels of drug are linked to adverse and overtly toxic effects. It is desirable to maintain blood levels of drug within a therapeutic window.

### SUMMARY OF THE INVENTION

Provided herein is a drug delivery composition comprising a lipophilic agent and a polymer wherein the lipophilic agent exhibits sustained release and wherein there is less than 10% agent release within the first hour of elution. Provided herein is a drug delivery composition comprising a lipophilic agent and a polymer wherein the elution profile is substantially linear, and wherein there is less than 10% agent release within the first hour of elution. In some embodiments, the elution profile is determined through in-vitro testing. In some embodiments, the elution profile is determined through in-vivo testing. In some embodiments, sustained release comprises the lipophilic agent releasing over a period of >3 hours, >24 hours, >1 week, >3 months, >6 months, and/or >12 months. In some embodiments, there is less than 5% agent release within the first hour of elution. In some embodiments, no agent is delivered in the first hour of elution. In some embodiments, the in-vitro elution profile exhibits no burst of elution in the first hour of elution, in the first 3 hours of elution, in the first 6 hours of elution, in the first 9 hours of elution, in the first 12 hours of elution, in the first 18 hours of elution, and/or in the first day of elution. In some embodiments, the elution profile is substantially linear once a detectable amount of drug is eluted. Provided herein is a drug delivery composition comprising a lipophilic agent and a polymer wherein the PK profile varies only within 10% of an average PK profile for the composition. Provided herein is a drug delivery composition comprising a lipophilic agent and a polymer wherein the AUC (area under the curve which is the integral of the concentration-time curve after a single dose or in steady state) of the PK profile of the composition varies only within 10% of an average AUC for the composition. Provided herein is a drug delivery composition comprising a lipophilic agent and a polymer wherein the Cmax (which is the peak plasma concentration of a drug after administration) of the PK profile of the composition varies only within 10% of an average Cmax for the composition. Provided herein is a drug delivery composition comprising a lipophilic agent and a polymer wherein the Tmax (which is the time it takes to reach Cmax) of the PK profile varies only within 10% of an average Tmax for the composition. In some embodiments, the PK profile of the composition is linear such that the linear regression fit to the in-vivo PK data has a R̂2 value of >0.8, or such that the linear regression fit to the in-vitro PK data has a R̂2 value of >0.8. In some embodiments, the lipophilic agent comprises a limus drug, mTOR inhibitors, antibiotic agents, and/or immunosuppressive agents. In some embodiments, the polymer comprises any one or more of a linear polymer, branched polymer, dendritic polymer, liquid crystalline polymer, amorphous polymer, semi-crystalline polymer, block co-polymer, tri-block copolymer, graft copolymer, polymer blend, ionomeric polymers, absorbable polymer, and bio-polymer. In some embodiments, the polymer comprises a tri-block polymer with two hydrophilic chains connected by a hydrophobic chain. In some embodiments, the polymer comprises a tri-block polymer with two hydrophobic chains connected by a hydrophilic chain. In some embodiments, the polymer comprises a pluronic polymer. In some embodiments, the pluronic polymer comprises a biocompatible reverse thermosensitive polymer. In some embodiments, the polymer comprises a poloxamer or a poloxamine, or a combination thereof. In some embodiments, the polymer comprises poloxamer 407, poloxamer 338, poloxamer 118, poloxamine 1107 or poloxamine 1307, or a combination thereof. In some embodiments, the polymer comprises block copolymers, random copolymers, graft polymers, branched copolymers, or a combination thereof. In some embodiments, the polymer comprises polyoxyalkylene block copolymer. In some embodiments, the polymer comprises at least one purified inverse thermosensitive polymer selected from the group consisting of purified poloxamers and purified poloxamines. In some embodiments, the composition has a transition temperature of between about 10° C. and about 40° C. In some embodiments, the composition has a transition temperature of between about 15° C. and about 30° C. In some embodiments, the volume of said composition at physiological temperature is about 80% to about 120% of its volume below its transition temperature. In some embodiments, the volume of said composition at physiological temperature is about 80% to about 120% of its volume below its transition temperature; and said composition has a transition temperature of between about 10° C. and about 40° C. In some embodiments, the volume of said composition at physiological temperature is about 80% to about 120% of its volume below its transition temperature; and said composition has a transition temperature of between about 15° C. and about 30° C. In some embodiments, the volume of said composition at physiological temperature is about 80% to about 120% of its volume below its transition temperature; said composition has a transition temperature of between about 10° C. and about 40° C.; and said composition comprises at least one purified inverse thermosensitive polymer selected from the group consisting of poloxamers and poloxamines. In some embodiments, the volume of said composition at physiological temperature is about 80% to about 120% of its volume below its transition temperature; said composition has a transition temperature of between about 15° C. and about 30° C.; and said composition comprises at least one purified inverse thermosensitive polymer selected from the group consisting of poloxamers and poloxamines. In some embodiments, the composition comprises about 50% to about 35% of said inverse thermosensitive polymer. In some embodiments, the composition comprises about 5% to about 30% of said inverse thermosensitive polymer. In some embodiments, the inverse thermosensitive polymer has a polydispersity index from about 1.5 to about 1.0. In some embodiments, the inverse thermosensitive polymer has a polydispersity index from about 1.2 to about 1.0. In some embodiments, the composition is a gel at a first temperature and a liquid at a second temperature, wherein the second temperature is lower than the first temperature. In some embodiments, the composition is a gel at body temperature. In some embodiments, the first temperature is at most 5° C. higher, 3° C. higher, at most 2° C. higher, at most 1° C. higher, at most 0.5° C. higher, at most 0.3° C. higher, or at most 0.2° C. higher than the second temperature. In some embodiments, the composition at physiological conditions after 24 hours contains >5% aqueous mass. In some embodiments, the composition has a sol (water) fraction of a hydrogel at physiological conditions after 24 hours. In some embodiments, the sol fraction is the fractional increase in the weight of the composition due to water absorption. In some embodiments, the composition is a hydrogel and/or has hydrogel properties. In some embodiments, at least a portion of the lipophilic agent is crystalline or semi-crystalline. In some embodiments, at least 50%, 60%, 70%, 75%, 80%, 85%, 90%, 92%, 95%, 98%, 99%, 99.0%, 99.5%, or 99% of the agent in the composition is in crystalline or semi-crystalline form. In some embodiments, the composition is formulated for use in, treatment of, or diagnosis of: chronic diseases; cancer; neurologic ailments, conditions, disorders, or diseases; pain, reproductive ailments, conditions, disorders, or diseases, or as part of a reproductive therapies or treatment; opthamalogic ailments, conditions, disorders, or diseases; GI ailments, conditions, disorders, or diseases; orthopedic ailments, conditions, disorders, or diseases; surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments, the composition is incorporated into a system comprising a substrate that carries the composition to the administration site or delivery site or treatment site. In some embodiments, the substrate comprises a medical implant, diagnostic device, interventional device, and/or surgical tool. In some embodiments, the substrate is a device, tool, or implant for use in orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments, the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology. Provided herein is a drug delivery composition comprising a biological agent and a polymer wherein the biological agent exhibits sustained release and wherein there is less than 10% agent release within the first hour of elution. Provided herein is a drug delivery composition comprising a biological agent and a polymer wherein the elution profile is substantially linear, and wherein there is less than 10% agent release within the first hour of elution. In some embodiments, the elution profile is determined through in-vitro testing. In some embodiments, the elution profile is determined through in-vivo testing. In some embodiments, sustained release comprises the biological agent releasing over a period of >3 hours, >24 hours, >1 week, >3 months, >6 months, and/or >12 months. In some embodiments, there is less than 5% agent release within the first hour of elution. In some embodiments, no agent is delivered in the first hour of elution. In some embodiments, the in-vitro elution profile exhibits no burst of elution in the first hour of elution, in the first 3 hours of elution, in the first 6 hours of elution, in the first 9 hours of elution, in the first 12 hours of elution, in the first 18 hours of elution, and/or in the first day of elution. In some embodiments, the elution profile is substantially linear once a detectable amount of agent is eluted. Provided herein is a drug delivery composition comprising a biological agent and a polymer wherein the PK profile varies only within 10% of an average PK profile for the composition. Provided herein is a drug delivery composition comprising a biological agent and a polymer wherein the AUC (area under the curve which is the integral of the concentration-time curve after a single dose or in steady state) of the PK profile of the composition varies only within 10% of an average AUC for the composition. Provided herein is a drug delivery composition comprising a biological agent and a polymer wherein the Cmax (which is the peak plasma concentration of a drug after administration) of the PK profile of the composition varies only within 10% of an average Cmax for the composition. Provided herein is a drug delivery composition comprising a biological agent and a polymer wherein the Tmax (which is the time it takes to reach Cmax) of the PK profile varies only within 10% of an average Tmax for the composition. In some embodiments, the PK profile of the composition is linear such that the linear regression fit to the in-vivo PK data has a R̂2 value of >0.8, or such that the linear regression fit to the in-vitro PK data has a R̂2 value of >0.8. In some embodiments, the biological agent comprises peptides, proteins, enzymes, derivatives and analogs of natural peptides, proteins and enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates, gene therapies, RNA, siRNA, and/or cellular therapies (for non-limiting example, stem cells or T-cells), or combinations thereof. In some embodiments, the composition is a gel at a first temperature and a liquid at a second temperature, wherein the second temperature is lower than the first temperature. In some embodiments, the first temperature is at most 5° C. higher, 3° C. higher, at most 2° C. higher, at most 1° C. higher, at most 0.5° C. higher, at most 0.3° C. higher, or at most 0.2° C. higher than the second temperature. In some embodiments, the composition is a gel at body temperature. In some embodiments, the polymer comprises any one or more of a linear polymer, branched polymer, dendritic polymer, liquid crystalline polymer, amorphous polymer, semi-crystalline polymer, block co-polymer, tri-block copolymer, graft copolymer, polymer blend, ionomeric polymers, absorbable polymer, and bio-polymer. In some embodiments, the polymer comprises a tri-block polymer with two hydrophilic chains connected by a hydrophobic chain. In some embodiments, the polymer comprises a tri-block polymer with two hydrophobic chains connected by a hydrophilic chain. In some embodiments, the polymer comprises a pluronic polymer. In some embodiments, the pluronic polymer comprises a biocompatible reverse thermosensitive polymer. In some embodiments, the polymer comprises a poloxamer or a poloxamine, or a combination thereof. In some embodiments, the polymer comprises poloxamer 407, poloxamer 338, poloxamer 118, poloxamine 1107 or poloxamine 1307, or a combination thereof. In some embodiments, the polymer comprises block copolymers, random copolymers, graft polymers, branched copolymers, or a combination thereof. In some embodiments, the polymer comprises polyoxyalkylene block copolymer. In some embodiments, the polymer comprises at least one purified inverse thermosensitive polymer selected from the group consisting of purified poloxamers and purified poloxamines. In some embodiments, the composition has a transition temperature of between about 10° C. and about 40° C. In some embodiments, the composition has a transition temperature of between about 15° C. and about 30° C. In some embodiments, the volume of said composition at physiological temperature is about 80% to about 120% of its volume below its transition temperature. In some embodiments, the volume of said composition at physiological temperature is about 80% to about 120% of its volume below its transition temperature; and said composition has a transition temperature of between about 10° C. and about 40° C. In some embodiments, the volume of said composition at physiological temperature is about 80% to about 120% of its volume below its transition temperature; and said composition has a transition temperature of between about 15° C. and about 30° C. In some embodiments, the volume of said composition at physiological temperature is about 80% to about 120% of its volume below its transition temperature; said composition has a transition temperature of between about 10° C. and about 40° C.; and said composition comprises at least one purified inverse thermosensitive polymer selected from the group consisting of poloxamers and poloxamines. In some embodiments, the volume of said composition at physiological temperature is about 80% to about 120% of its volume below its transition temperature; said composition has a transition temperature of between about 15° C. and about 30° C.; and said composition comprises at least one purified inverse thermosensitive polymer selected from the group consisting of poloxamers and poloxamines. In some embodiments, the composition comprises about 50% to about 35% of said inverse thermosensitive polymer. In some embodiments, the composition comprises about 5% to about 30% of said inverse thermosensitive polymer. In some embodiments, the inverse thermosensitive polymer has a polydispersity index from about 1.5 to about 1.0. In some embodiments, the inverse thermosensitive polymer has a polydispersity index from about 1.2 to about 1.0. In some embodiments, the composition at physiological conditions after 24 hours contains >5% aqueous mass. In some embodiments, the composition has a sol (water) fraction of a hydrogel at physiological conditions after 24 hours. In some embodiments, the sol fraction is the fractional increase in the weight of the composition due to water absorption. In some embodiments, the composition is a hydrogel and/or has hydrogel properties. In some embodiments, the biological agent is in an active form, wherein active form comprises having a degree of secondary, tertiary and/or quaternary structure upon which the activity of the agent depends. In some embodiments, at least 50%, 60%, 70%, 75%, 80%, 85%, 90%, 92%, 95%, 98%, 99%, 99.0%, 99.5%, or 99% of the agent in the composition is in active form. In some embodiments, the composition is formulated for use in, treatment of, or diagnosis of: chronic diseases; cancer; neurologic ailments, conditions, disorders, or diseases; pain, reproductive ailments, conditions, disorders, or diseases, or as part of a reproductive therapies or treatment; opthamalogic ailments, conditions, disorders, or diseases; GI ailments, conditions, disorders, or diseases; orthopedic ailments, conditions, disorders, or diseases; surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments, the composition is incorporated into a system comprising a substrate that carries the composition to the administration site or delivery site or treatment site. In some embodiments, the substrate comprises a medical implant, diagnostic device, interventional device, and/or surgical tool. In some embodiments, the substrate is a device, tool, or implant for use in orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments, the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology. Provided herein is a drug delivery composition comprising a lipophilic agent and a polymer, wherein said composition is a hydrogel, wherein the lipophilic agent exhibits sustained release, and wherein there is less than 35% lipophilic agent release within the first hour of elution. Provided herein is a drug delivery composition comprising a lipophilic agent and a polymer, wherein said composition is a hydrogel, wherein the elution profile is substantially linear, and wherein there is less than 35% lipophilic agent release within the first hour of elution. In some embodiments, the elution profile is determined through in-vitro testing. In some embodiments, the elution profile is determined through in-vivo testing. In some embodiments, sustained release comprises the lipophilic agent releasing over a period of >24 hours, >3 days, >1 week, >10 days, or 16 days. In some embodiments, there is less than 10% agent release within the first hour of elution. In some embodiments, no agent is delivered in the first hour of elution. In some embodiments, the in-vitro elution profile exhibits no burst of elution in the first hour of elution, in the first 3 hours of elution, in the first 6 hours of elution, in the first 9 hours of elution, in the first 12 hours of elution, in the first 18 hours of elution, and/or in the first day of elution. In some embodiments, the elution profile is substantially linear once a detectable amount of drug is eluted. In some embodiments, the lipophilic agent comprises a limus drug, mTOR inhibitors, antibiotic agents, and/or immunosuppressive agents. In some embodiments, the lipophilic agent comprises a limus drug selected from rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus. In some embodiments, the lipophilic agent is rapamycin. In some embodiments, the polymer comprises any one or more of a linear polymer, branched polymer, dendritic polymer, liquid crystalline polymer, amorphous polymer, semi-crystalline polymer, block co-polymer, tri-block copolymer, graft copolymer, polymer blend, ionomeric polymers, absorbable polymer, and bio-polymer. In some embodiments, at least a portion of the lipophilic agent is crystalline or semi-crystalline. In some embodiments, at least 50%, 60%, 70%, 75%, 80%, 85%, 90%, 92%, 95%, 98%, 99%, 99.0%, 99.5%, or 99% of the lipophilic agent in the composition is in crystalline or semi-crystalline form. In some embodiments is a drug delivery composition formulated for use in, treatment of, or diagnosis of: chronic diseases; cancer; neurologic ailments, conditions, disorders, or diseases; pain, reproductive ailments, conditions, disorders, or diseases, or as part of a reproductive therapies or treatment; opthamalogic ailments, conditions, disorders, or diseases; GI ailments, conditions, disorders, or diseases; orthopedic ailments, conditions, disorders, or diseases; surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments is a drug delivery composition wherein the composition is incorporated into a system comprising a substrate that carries the composition to the administration site or delivery site or treatment site. In some embodiments, the substrate comprises a medical implant, diagnostic device, interventional device, and/or surgical tool. In a further embodiment, the substrate is a device, tool, or implant for use in orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments, the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the cumulative fraction release of rapamycin from three hydrogel compositions prepared in Example 1.

FIG. 1B depicts the cumulative mass release of rapamycin from three hydrogel compositions prepared in Example 1 using the zero order model.

FIG. 2 depicts the log cumulative percent release of rapamycin from three hydrogel compositions prepared in Example 1 using the Korsmeyer-Peppas model.

FIG. 3 depicts the cumulative fraction release of rapamycin from three hydrogel compositions prepared in Example 1 using the Higuchi model.

### DETAILED DESCRIPTION OF THE INVENTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof. This application relates to U.S. Provisional Application No. 61/226,239 filed Jul. 16, 2009; U.S. Provisional Application No. 61/081,691, filed Jul. 17, 2008, and U.S. Provisional Application No. 61/212,964, filed Apr. 17, 2009. The contents of these applications are incorporated herein by reference in their entirety.

### DEFINITIONS

As used in the present specification, the following words and phrases are generally intended to have the meanings as set forth below, except to the extent that the context in which they are used indicates otherwise.

“Drug delivery composition” as used herein refers to a composition capable of delivering a drug when administered to a subject independently of a substrate such as a stent or other medical device coated with the composition. Once the composition is administered to the subject, the composition is separated from the device used to administer the composition (e.g., a syringe) and drug delivery is carried out by the drug delivery composition without the need for a substrate. It should be noted that embodiments described herein and claimed below when reference is not made to “drug delivery composition” include compositions that are delivered as part of a device such as a transdermal delivery device.

Examples of pharmaceutical agents employed in conjunction with the invention include, rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

The pharmaceutical agents may, if desired, also be used in the form of their pharmaceutically acceptable salts or derivatives (meaning salts which retain the biological effectiveness and properties of the compounds of this invention and which are not biologically or otherwise undesirable), and in the case of chiral active ingredients it is possible to employ both optically active isomers and racemates or mixtures of diastereoisomers. As well, the pharmaceutical agent may include a prodrug, a hydrate, an ester, a polymorph, a derivative or analogs of a compound or molecule.

The pharmaceutical agent may be an antibiotic agent, as described herein.

“Prodrugs” are derivative compounds derivatized by the addition of a group that endows greater solubility to the compound desired to be delivered. Once in the body, the prodrug is typically acted upon by an enzyme, e.g., an esterase, amidase, or phosphatase, to generate the active compound.

An “anti-cancer agent”, “anti-tumor agent” or “chemotherapeutic agent” refers to any agent useful in the treatment of a neoplastic condition. There are many chemotherapeutic agents available in commercial use, in clinical evaluation and in pre-clinical development that are useful in the devices and methods of the present invention for treatment of cancers.

“Active agent” as used herein comprises any therapeutic agent including a pharmaceutical agent which may be lipophilic agent as described herein or a biologic agent as described herein.

A “lipophilic agent” or “lipophilic compound” as used herein include poorly water soluble pharmaceutical agents. Lipophilic agents tend to combine with or dissolve in lipids or fats. In some embodiments, lipophilic agents may also be hydrophobic. In other embodiments, the lipophilic agent may not be hydrophobic. The lipophilic agents may be only partially lipophilic, meaning that, for example, only a portion of the agents has lipophilic properties, or that the agent has some lipophilic properties, but also may exhibit other properties such as hydrophilicity, or that the agent has a low degree of lipophilicity. The agents referred to herein as lipophilic agents have a hydrophilic-lipophilic balance (HLB) of less than 10, 6 or less, or less than 6, and fall within the HLB International scale, which ranges from 0-20.

In some embodiments, Lipophilic agents comprise, for non-limiting example: anticancer agents, immunosuppressive agents, corticoids, analgesics, human-immunodeficiency-virus protease inhibitors, antibiotic agents, or combinations thereof. In some embodiments, Lipophilic anti-cancer agents comprise, for non-limiting example: doxorubicin, daunorubicin, idarubicin, vinblastine, vincristine, etoposide, methotrexate, or combinations thereof. In some embodiments, Lipophilic corticoids comprise, for non-limiting example: dexamethasone, hydrocortisone, or combinations thereof. In some embodiments, Lipophilic analgesics comprise, for non-limiting example: morphine, or fentanyl or combinations thereof. In some embodiments, Human-immunodeficiency-virus protease inhibitors comprise, for non-limiting example: amprenavir, saquinavir, ritonavir, or combinations thereof. Farglitazar, for example, is partly lipophilic and partly hydrophilic, and may be considered a lipophilic agent herein, as would other therapeutic or biological agents that are partly lipophilic. Other agents may be used as noted elsewhere herein, or as known to one of skill in the art, so long the agent demonstrates lipophilic properties (e.g. HLB of less than 10).

In some embodiments, Lipophilic agents may comprise antibiotic agents or macrolide antibiotics such as: antierythromycin base and its pharmaceutically acceptable salts and esters, as well as the semisynthetic derivatives of erythromycin, including but not limited to 6-O-methyl-erythromycin (clarithromycin), the erythromycin 9-oximes, erythromycin 11,12-cyclic carbamates and 4″-deoxy-11,12 carbamates, 1-0-methyl-, and 6,11-di-O-methyl erythromycins, 8-fluoroethyromycin, erythromycin 4″-carbamates, and compounds having various combinations of these structural modifications, as well as their pharmaceutically acceptable salts and esters.

In some embodiments, “immunosuppressive agents” are used. Immunosuppressive agents may also be referred to as immunosupressants or immunosuppressive drugs or immunomodulators or immunomodulating drugs. Such immunosuppressive agents may include a cyclosporine, or ascomycine or their immunosuppressive analogs or derivatives, e.g. cyclosporin A, cyclosporin G, FK-506, ABT-281, ASM 981; an mTOR inhibitor, e.g. rapamycin, 40-O-(2-hydroxy)ethyl-rapamycin. Example immunosupressants include, but are not limited to: antimetabolites such as purine synthesis inhibitors (e.g. azathioprine, mycophenolic acid), pyrimidine synthesis inhibitors (e.g. leflunomide and teriflunomide), and antifolate (e.g. methotrexate); macrolides or other IL-2 inhibitors such as FKBP/cyclopholin/calcineurin (e.g. tacrolimus, ciclosporin, pimecrolimus, abetimus or gusperimus); or TNF inhibitors such as thalidomide or lenalidomide. Other example immunosupressants include, but are not limited to: IL-1 receptor antagonists such as anakinra, or mTOR inhibitors (sirolimus, everolimus, ridaforolimus, temserolimus, umirolimus, zotarolims). Other example immunosupressants include, but are not limited to: monoclonoal antibodies having a non-cellular serum target such as: complement component 5 (Eculizumab), TNFs (Infliximab, Adalimumab, Certolizumab pegol, Afelimomab, Golimumab), Interleukin 5 (Mepolizumab), Immunoglobulin E (Omalizumab), BAYX (Nerelimomab), Interferon (Faralimomab), IL-6 (Elsilimomab), or IL-12 and IL-23 (Lebrikizumab, Ustekinumab). Other example immunosupressants include, but are not limited to: monoclonoal antibodies having a cellular target such as: CD3 (Muromonab-CD3, Otelixizumab, Teplizumab, Visilizumab), CD4 (Clenoliximab, Keliximab, Zanolimumab), CD11a (Efalizumab), CD18 (Erlizumab), CD20 (Afutuzumab, Rituximab, Ocrelizumab, Pascolizumab), CD23 (Gomiliximab, Lumiliximab), CD40 (Teneliximab, Toralizumab), CD62L/L-selectin (Aselizumab), CD80 (Galiximab), CD147/Basigin (Gavilimomab), CD154 (Ruplizumab), BLyS (Belimumab), CTLA-4 (Ipilimumab, Tremelimumab), CAT (Bertilimumab, Lerdelimumab, Metelimumab), Integrin (Natalizumab), Interleukin-6 receptor (Tocilizumab), LFA-1 (Odulimomab), IL-2 receptor/CD25 (Basiliximab, Daclizumab, Inolimomab), or T-lymphocyte (Zolimomab aritox). Other example immunosupressants include, but are not limited to: monoclonal antibodies (unsorted target) such as: Atorolimumab, Cedelizumab, Fontolizumab, Maslimomab, Morolimumab, Pexelizumab, Reslizumab, Rovelizumab, Siplizumab, Talizumab, Telimomab aritox, Vapaliximab, or Vepalimomab. Other example immunosupressants include, but are not limited to: polyclonal antibodies such as Anti-thymocyte globulin, or Anti-lymphocyte globulin. Other example immunosupressants include, but are not limited to: CTLA-4 (Abatacept, Belatacept), TNF inhibitor, (Etanercept, Pegsunercept), Aflibercept, Alefacept, or Rilonacept.

“mTOR inhibitor” as used herein includes, but is not limited to rapamycin (sirolimus) or a salt, prodrug, ester, or derivative thereof. Rapamycin is a known macrolide antibiotic, also known as Sirolimus & Rapamune is an mTOR inhibitor. Rapamycin inhibits cell motility by suppression of mTOR-mediated pathways. mTOR inhibitors such as rapamycin and its analogs are lipophilic. Rapamycin and it's analogs (or rapalogues e.g. CCI-779) modulate the phosphatidylinositol 3-kinase (PI3K) signalling cascade by binding to an allosteric site on the mTORC1 complex. Mammalian target of rapamycin (mTOR), a class IV PI3K protein kinase, forms two functional complexes, mTORC1 and mTORC2. mTOR plays a major role in regulating cell growth and proliferation and it's aberrant function is implicated in a number of cancers.

Suitable derivatives may include those disclosed in WO94/09010, WO96/16691, WO96/41807, U.S. Pat. No. 6,362,718 or WO99/15530, which are included herein by reference. Certain rapamycin derivatives include 32-deoxorapamycin, 16-pent-2-ynyloxy-32-deoxorapamycin, 16-pent-2-ynyloxy-32(S)-dihydro-rapamycin, 16-pent-2-ynyloxy-32(S)-dihydro-40-O-(2-hydroxyethyl)-rapamycin and, more preferably, 40-O-(2-hydroxyethyl)rapamycin. Further examples of rapamycin derivatives include e.g. CCI779 or 40-[3-hydroxy-2-(hydroxymethyl)-2-methylpropanoate]-rapamycin or a pharmaceutically acceptable salt thereof, as disclosed in U.S. Pat. No. 5,362,718, ABT578 or 40-(tetrazolyl)-rapamycin, particularly 40-epi-(tetrazolyl)-rapamycin, e.g. as disclosed in WO 99/15530, or rapalogs as disclosed e.g. in WO 98/02441 and WO01/14387, e.g. AP23573 or TAFA-93. Another example of an mTOR inhibitor includes BEZ235 (NVP-BEZ235) which is a dual ATP-competitive phosphatidylinositol 3-kinase (PI3K) and mammalian target of rapamycin (mTOR) inhibitor of p110α, p110γ, p110δ and p110β with IC50 of 4 nM, 5 nM, 7 nM and 75 nM, respectively. Another example of an mTOR inhibitor includes Everolimus (RAD001), also known as SDZ-RAD, Certican, Zortress and Afinitorm, and is an mTOR inhibitor with IC50 of 0.63 nM. Another example of an mTOR inhibitor includes PI-103 is a potent, cell-permeable, ATP-competitive PI3K family member inhibitor with IC50 of 2, 8, 20, 26, 48, 83, 88, 150 nM for DNA-PK, p110α, mTORC1, PI3-KC2β, p110δ, mTORC2, p110β, and p110γ, respectively. Another example of an mTOR inhibitor includes Temsirolimus (Torisel). AZD8055 is a potent, selective, and orally bioavailable ATP-competitive mTOR kinase inhibitor with an IC50 of 0.8 nM. Another example of an mTOR inhibitor includes AZD8055 is a potent, selective, and orally bioavailable ATP-competitive mTOR kinase inhibitor with an IC50 of 0.8 nM. Another example of an mTOR inhibitor includes Ku-0063794 is an mTOR inhibitor, IC50˜10 nM for mTORC1 and mTORC2, respectively. Another example of an mTOR inhibitor includes NVP-BGT226 is a novel dual PI3K/mTOR inhibitor with an IC50 of 1 nM. Another example of an mTOR inhibitor includes PF-04691502 is a potent and selective dual PI3K/mTOR inhibitor to phosphorylation of AKT T308 and AKT S473 with IC50 of 7.5 and 3.8 nM, respectively. Another example of an mTOR inhibitor includes CH5132799 is a novel and selective class I PI3K inhibitor with IC50 of 0.014, 0.12, 0.5, 0.036, 5.3 and 1.6 μM for PI3Kα, PI3Kβ, PI3Kδ, PI3Kγ, PI3K C2β and mTOR, respectively. Another example of an mTOR inhibitor includes GDC-0980 (RG7422) is a selective, dual PI3 Kinase and mTOR inhibitor with IC50 of 5, 27, 7, and 14 nM for PI3Kα, β, δ, and γ, respectively. Another example of an mTOR inhibitor includes WAY-600 is a potent ATP-competitive mTOR inhibitor with an IC50 of 9 nM. Another example of an mTOR inhibitor includes WYE-125132 is a highly potent, ATP-competitive and specific mTOR kinase inhibitor with an IC50 of 0.19 nM. Another example of an mTOR inhibitor includes WYE-687 is a potent ATP-competitive mTOR inhibitor with an IC50 of 7 nM. Another example of an mTOR inhibitor includes GSK2126458 is a highly potent PI3K and mTOR inhibitor with an app Ki of 19 μM for PI3K. Another example of an mTOR inhibitor includes PKI-587 is a highly potent dual PI3K/mTOR kinase inhibitor with IC50 of 0.4 nM and <0.1 μM for PI3K-α and mTOR, respectively. Another example of an mTOR inhibitor includes PP121 is a multitargeted dual receptor tyrosine kinases inhibitor with IC50 of 0.052, 1.4, 0.15, 1.1, 0.06, 0.01 and 0.002 μM for p110α, p110β, p110δ, p110γ, DNA-PK, mTOR and PDGFR, Another example of an mTOR inhibitor includes OSI027 is a potent mammalian target of rapamycin (mTOR) kinase inhibitor. respectively. Another example of an mTOR inhibitor includes Palomid 529 (P529) is a novel PI3K/Akt/mTOR inhibitor with a GI50 of <35 μM in the NCI-60 cell lines panel. PP242 is a novel selective mTOR inhibitor with an IC50 of 8 nM. Another example of an mTOR inhibitor includes Chrysophanic acid (Chrysophanol) is a EGFR/mTOR pathway inhibitor. XL765 is a mixed mTOR/PI3k inhibitor with IC50 of 157, 39, 113, 9 and 43 nM for mTOR, p110α, β, γ and δ, respectively. Another example of an mTOR inhibitor includes GSK1059615 is a pan-PI3K reversible inhibitor, IC50:PI3Kα (0.4 nM), β (0.6 nM), γ (5 nM), δ (2 nM) and mTOR (12 nM). Another example of an mTOR inhibitor includes WYE-354 is an mTOR inhibitor with an IC50 of 5 nM. Deforolimus (Ridaforolimus) is a small-molecule inhibitor of mTOR.

“‘limus drugs” or “’limus agents” are macrolide immunosuppressive drugs. They include, for nonlimiting example, sirolimus, everolimus, biolimus A9, zotarolimus, tacrolimus and pimecrolimus. Sirolimus, everolimus, biolimus A9 (biolimus), and zotarolimus all bind to the FKBP12 binding protein, which subsequently binds to the mammalian target of rapamycin (mTOR) and thereby blocks the cell cycle mainly of the smooth muscle cell from the G1 to S phase. The mechanisms of action of tacrolimus and pimecrolimus are different. Both drugs bind to FKBP506. The tacrolimus/pimecrolimus FKBP506 complex subsequently inhibits the calcineurin receptor, which leads to decreased cytokine expression on the cell surface membrane and results in an inhibition of T-cell activation and lower smooth muscle cell selectivity.

Sirolimus, a natural macrocyclic lactone that is able to inhibit mTOR. Sirolimus possess potent antiproliferative and immunosuppressive effects.

Everolimus is a sirolimus analog with a single minimal alteration in its molecular structure (position 40), without a chemical modification of the mTOR binding domain.

Zotarolimus also has a change on position 40, and is known as ABT-578, Abbott Pharmaceuticals, Abbott Park, Ill. It likewise contains antiproliferative and antiinflammatory effects, but zotarolimus is suggested to have higher tissue retention when delivery in a drug eluting stent as compared with the sirolimus eluting stent.

Biolimus A9 is a highly lipophilic sirolimus analog that inhibits T cell and smooth muscle cell proliferation.

Although a part of the ‘limus family, pimecrolimus does not block mTOR and inhibits to a much lesser degree the endothelial cell proliferation. The active pharmaceutical ingredient of pimecrolimus is Elidel, an FDA-approved drug developed by Novartis Pharmaceuticals Corp, East Hanover, N.J.) for the treatment of atopic dermatitis.

Although a part of the Limus family, pimecrolimus does not block mTOR and inhibits to a much lesser degree the endothelial cell proliferation. The active pharmaceutical ingredient of pimecrolimus is Elidel, an FDA-approved drug developed by Novartis Pharmaceuticals Corp, East Hanover, N.J.) for the treatment of atopic dermatitis.

“Macrolide immunosuppressive (limus) drug” or “macrolide immunosuppressive (limus) agent” may include, for non-limiting example: one or more of: rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

“Macrolide antibiotic drug” or “macrolide antibiotic” may include, for non-limiting example: Azithromycin, Clarithromycin, Dirithromycin, Erythromycin, Roxithromycin, Telithromycin, Carbomycin A, Josamycin, Kitasamycin, Midecamycin/midecamycin acetate, Oleandomycin, Solithromycin, Spiramycin, Troleandomycin, or Tylosin/tylocine, their pharmaceutically acceptable salts, esters, derivatives, isomers, racemates, diastereoisomers, prodrugs, hydrates, or analogs thereof. Among the more common pharmaceutically acceptable salts and esters of macrolide antibiotics are the acetate, estolate (lauryl sulfate salt of the propionate ester), ethyl succinate, gluceptate (glucoheptonate), lactobionate, stearate, and hydrochloride forms.

By “pharmaceutically acceptable salts and esters” or “salts” or “esters” as used herein can mean those salts and esters which are, within the scope of sound medical judgment, suitable for use in contact with the tissues of humans and lower animals without undue toxicity, irritation, allergic response, and the like, commensurate with a reasonable benefit/risk ratio, and effective for their intended use. Other acid salts used in the pharmaceutical arts are the following: adipate, alginate, aspartate, benzoate, benzene sulfonate, bisulfate, butyrate, citrate, camphorate, camphorsulfonate, cyclopentanepropionate, digluconate, dodecylsulfate, ethanesulfonate, fumarate, gluconate, glycerophosphate, hemisulfate, heptonate, hexanoate, hydrobromide, hydroiodide, 2-hydroxy ethanesulfonate, lactate, maleate, methanesulfonate, 2-naphthalenesulfonate, nicotinate, oxalate, pamoate, pantothenate, pectinate, persulfate, 3-phenylpropionate, picrate, pivalate, propionate, succinate, tartrate, thiocyanate, tosylate, and undecanoate. Basic nitrogen-containing groups can be quaternized with such agents as lower alkyl halides, such as methyl, ethyl, propyl and butyl chloride, bromides and iodides; dialkyl sulfates like dimethyl, diethyl, dibutyl, and diamyl sulfates; long chain halides such as decyl, lauryl, myristyl and stearyl chlorides, bromides and iodides; aralkyl halides like benzyl and phenethyl bromides and others. Water or oil-soluble or dispersible products are thereby obtained.

The lipophilic agents may include: antiinfectives such as antibiotics and antiviral agents; analgesics and analgesic combinations; anorexics; antihelmintics; antiarthritics; antiasthmatic agents; anticonvulsants; antidepressants; antidiuretic agents; antidiarrheals; antihistamines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and/or tranquilizers. Suitable pharmaceuticals for parenteral administration are well known as is exemplified by the Handbook on Injectable Drugs, 6.sup.th Edition, by Lawrence A. Trissel, American Society of Hospital Pharmacists, Bethesda, Md., 1990 (hereby incorporated by reference).

The compositions described herein may comprise biological agents rather than lipophilic agents or in addition to the lipophilic agent. Such biological agents may comprise, for non-limiting example: proteins, vaccines, peptides, polypeptides, polynucleotides, nucleoproteins, polysaccharides, glycoproteins, lipoproteins, hormones, sRNA, etc., including synthetic and biologically engineered analogs thereof, whether or not the biological agents themselves have lipophilic properties.

A “biological agent” or “active biological agent” as used herein refers to a substance, originally produced by living organisms, that can be used to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). It is possible that the biological agents of the invention may also comprise two or more active biological agents or an active biological agent combined with a pharmaceutical agent, a stabilizing agent or chemical or biological entity. Although the biological agent may have been originally produced by living organisms, those of the present invention may also have been synthetically prepared, or by methods combining biological isolation and synthetic modification. By way of a non-limiting example, a nucleic acid could be isolated form from a biological source, or prepared by traditional techniques, known to those skilled in the art of nucleic acid synthesis. Furthermore, the nucleic acid may be further modified to contain non-naturally occurring moieties. Non-limiting examples of biological agents include peptides, proteins, enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates and the like. They further include, but are not limited to, antirestenotic agents, antidiabetics, analgesics, antiinflammatory agents, antirheumatics, antihypotensive agents, antihypertensive agents, psychoactive drugs, tranquilizers, antiemetics, muscle relaxants, glucocorticoids, agents for treating ulcerative colitis or Crohn's disease, antiallergics, antibiotics, antiepileptics, anticoagulants, antimycotics, antitussives, arteriosclerosis remedies, diuretics, proteins, peptides, enzymes, enzyme inhibitors, gout remedies, hormones and inhibitors thereof, cardiac glycosides, immunotherapeutic agents and cytokines, laxatives, lipid-lowering agents, migraine remedies, mineral products, otologicals, anti parkinson agents, thyroid therapeutic agents, spasmolytics, platelet aggregation inhibitors, vitamins, cytostatics and metastasis inhibitors, phytopharmaceuticals and chemotherapeutic agents. Preferably, the biological agent is a peptide, protein or enzyme, including derivatives and analogs of natural peptides, proteins and enzymes. The biological agent may also be a hormone, gene therapies, RNA, siRNA, and/or cellular therapies (for non-limiting example, stem cells or T-cells).

“Activity” as used herein refers to the ability of a pharmaceutical or active biological agent to prevent or treat a disease (meaning any treatment of a disease in a mammal, including preventing the disease, i.e. causing the clinical symptoms of the disease not to develop; inhibiting the disease, i.e. arresting the development of clinical symptoms; and/or relieving the disease, i.e. causing the regression of clinical symptoms). Thus the activity of a pharmaceutical or active biological agent should be of therapeutic or prophylactic value.

“Secondary, tertiary and quaternary structure” as used herein are defined as follows. The active biological agents of the present invention will typically possess some degree of secondary, tertiary and/or quaternary structure, upon which the activity of the agent depends. As an illustrative, non-limiting example, proteins possess secondary, tertiary and quaternary structure. Secondary structure refers to the spatial arrangement of amino acid residues that are near one another in the linear sequence. The .alpha.-helix and the .beta.-strand are elements of secondary structure. Tertiary structure refers to the spatial arrangement of amino acid residues that are far apart in the linear sequence and to the pattern of disulfide bonds. Proteins containing more than one polypeptide chain exhibit an additional level of structural organization. Each polypeptide chain in such a protein is called a subunit. Quaternary structure refers to the spatial arrangement of subunits and the nature of their contacts. For example hemoglobin consists of two .alpha. and two .beta. chains. It is well known that protein function arises from its conformation or three dimensional arrangement of atoms (a stretched out polypeptide chain is devoid of activity). Thus one aspect of the present invention is to manipulate active biological agents, while being careful to maintain their conformation, so as not to lose their therapeutic activity.

In some embodiments the drug delivery composition may be incorporated into a system comprising a substrate that carries the composition to the administration site or delivery site or treatment site. The substrate may remain with the composition upon administration (or upon delivery of the composition) and for any amount of time or indefinitely thereafter, or be removed upon administration (or upon delivery of the composition) leaving the composition at the administration site or delivery site or treatment site.

“Substrate” as used herein, refers to any surface upon which it is desirable to deposit a coating comprising a polymer and a pharmaceutical or biological agent, wherein the coating process does not substantially modify the morphology of the pharmaceutical agent or the activity of the biological agent. Biomedical implants are of interest for the present invention; however the present invention is not intended to be restricted to this class of substrates. Interventional devices are also of interest for the present invention; however the present invention is not intended to be restricted to this class of substrates. Diagnostic devices are also of interest for the present invention; however the present invention is not intended to be restricted to this class of substrates. Those of skill in the art will appreciate alternate substrates that could benefit from the coating process described herein, such as pharmaceutical tablet cores, as part of an assay apparatus or as components in a diagnostic kit (e.g. a test strip).

Substrates contemplated herein include, for non-limiting example: medical implants, diagnostic devices, interventional devices, and/or surgical tools. Substrates contemplated herein include, for non-limiting example any device, tool, or implant for use in: orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications.

In some embodiments, the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology.

A “Biomedical implant” as used herein refers to any implant for insertion into the body of a human or animal subject, including but not limited to stents (e.g., coronary stents, vascular stents including peripheral stents and graft stents, urinary tract stents, urethral/prostatic stents, rectal stent, oesophageal stent, biliary stent, pancreatic stent), electrodes, catheters, leads, implantable pacemaker, cardioverter or defibrillator housings, joints, screws, rods, ophthalmic implants, femoral pins, bone plates, grafts, anastomotic devices, perivascular wraps, sutures, staples, shunts for hydrocephalus, dialysis grafts, colostomy bag attachment devices, ear drainage tubes, leads for pace makers and implantable cardioverters and defibrillators, vertebral disks, bone pins, suture anchors, hemostatic barriers, clamps, screws, plates, clips, vascular implants, tissue adhesives and sealants, tissue scaffolds, various types of dressings (e.g., wound dressings), bone substitutes, intraluminal devices, vascular supports, etc. In some embodiments, the substrate is selected from the group consisting of: stents, joints, screws, rods, pins, plates, staples, shunts, clamps, clips, sutures, suture anchors, electrodes, catheters, leads, grafts, dressings, pacemakers, pacemaker housings, cardioverters, cardioverter housings, defibrillators, defibrillator housings, prostheses, ear drainage tubes, ophthalmic implants, orthopedic devices, vertebral disks, bone substitutes, anastomotic devices, perivascular wraps, colostomy bag attachment devices, hemostatic barriers, vascular implants, vascular supports, tissue adhesives, tissue sealants, tissue scaffolds and intraluminal devices. The implant may be temporarily used in or permanently implanted in the body of a human or animal subject. The implant may only be used in a transient manner in or on the body of the subject, for non-limiting example: during a medical procedure that does not leave the implant in or on the subject once the medical procedure is completed.

The substrate may be formed from any suitable material, including but not limited to polymers (including stable or inert polymers, organic polymers, organic-inorganic copolymers, inorganic polymers, and biodegradable polymers), metals, metal alloys, inorganic materials such as silicon, and composites thereof, including layered structures with a core of one material and one or more coatings of a different material. Substrates may be made of a conducting material facilitate electrostatic capture. However, the invention contemplates the use of electrostatic capture, as described below, in conjunction with substrate having low conductivity or which are non-conductive. To enhance electrostatic capture when a non-conductive substrate is employed, the substrate is processed for example while maintaining a strong electrical field in the vicinity of the substrate.

In some embodiments, the substrate comprises a stainless steel material. In some embodiments, the substrate comprises a material comprising a cobalt chromium alloy. In some embodiments, the substrate comprises a material comprising the following percentages by weight: about 0.05 to about 0.15 C, about 1.00 to about 2.00 Mn, about 0.04 Si, about 0.03 P, about 0.3 S, about 19.0 to about 21.0 Cr, about 9.0 to about 11.0 Ni, about 14.0 to about 16.00 W, about 3.0 Fe, and Bal. Co. In some embodiments, the implant comprises a material comprising at most the following percentages by weight: about 0.025 C, about 0.15 Mn, about 0.15 Si, about 0.015 P, about 0.01 S, about 19.0 to about 21.0 Cr, about 33 to about 37 Ni, about 9.0 to about 10.5 Mo, about 1.0 Fe, about 1.0 Ti, and Bal. Co. In some embodiments, the substrate comprises a material comprising L605 alloy. In some embodiments, the substrate comprises a material comprising a platinum chromium alloy instead of a cobalt-chromium alloy. In some embodiments, the substrate comprises a material comprising MP35N alloy. In some embodiments, the substrate comprises a material comprising the following percentages by weight: about 35 Ni, about 35 Cr, about 20 Co, and about 10 Mo. In some embodiments, the substrate comprises a material comprising a cobalt chromium nickel alloy. In some embodiments, the substrate comprises a material comprising Elgiloy.®/Phynox.®.In some embodiments, the substrate comprises a material comprising the following percentages by weight: about 39 to about 41 Co, about 19 to about 21 Cr, about 14 to about 16 Ni, about 6 to about 8 Mo, and Balance Fe. In some embodiments, the substrate comprises a material comprising a platinum chromium alloy. In some embodiments, the substrate comprises an alloy as described in U.S. Pat. No. 7,329,383 incorporated in its entirety herein by reference. In some embodiments, the substrate comprises an alloy as described in U.S. patent application Ser. No. 11/780,060 incorporated in its entirety herein by reference. In some embodiments, the substrate comprises a material comprising stainless steel, 316L stainless steel, BioDur.® 108 (UNS S29108), 304L stainless steel, and an alloy including stainless steel and 5-60% by weight of one or more radiopaque elements such as Pt, IR, Au, W, PERSS.® as described in U.S. Publication No. 2003/001830 incorporated in its entirety herein by reference, U.S. Publication No. 2002/0144757 incorporated in its entirety herein by reference, and U.S. Publication No. 2003/0077200 incorporated in its entirety herein by reference, nitinol, a nickel-titanium alloy, cobalt alloys, Elgiloy.®, L605 alloys, MP35N alloys, titanium, titanium alloys, Ti-6A1-4V, Ti-50Ta, Ti-10Ir, platinum, platinum alloys, niobium, niobium alloys, Nb-1Zr, Co-28Cr-6Mo, tantalum, and tantalum alloys. Other examples of materials that are comprised in the device (or substrate thereof) are described in U.S. Publication No. 2005/0070990 incorporated in its entirety herein by reference, and U.S. Publication No. 2006/0153729 incorporated in its entirety herein by reference. Other materials include elastic biocompatible metal such as superelastic or pseudo-elastic metal alloys, as described, for example in Schetsky, L. McDonald, “Shape Memory Alloys”, Encyclopedia of Chemical Technology (3d Ed), John Wiley & Sons 1982, vol. 20 pp. 726-736 incorporated herein by reference, and U.S. Publication No. 2004/0143317 incorporated in its entirety herein by reference.

Subjects into which biomedical implants of the invention may be applied or inserted include both human subjects (including male and female subjects and infant, juvenile, adolescent, adult and geriatric subjects) as well as animal subjects (including but not limited to pig, rabbit, mouse, dog, cat, horse, monkey, etc.) for veterinary purposes and/or medical research.

In a preferred embodiment the biomedical implant is an expandable intraluminal vascular graft or stent (e.g., comprising a wire mesh tube) that can be expanded within a blood vessel by an angioplasty balloon associated with a catheter to dilate and expand the lumen of a blood vessel, such as described in U.S. Pat. No. 4,733,665 to Palmaz.

In some embodiments, the substrate is an interventional device. An “interventional device” as used herein refers to any device for insertion into the body of a human or animal subject, which may or may not be left behind (implanted) for any length of time including, but not limited to, angioplasty balloons, cutting balloons.

In some embodiments, the substrate is a diagnostic device. A “diagnostic device” as used herein refers to any device for insertion into the body of a human or animal subject in order to diagnose a condition, disease or other of the patient, or in order to assess a function or state of the body of the human or animal subject, which may or may not be left behind (implanted) for any length of time.

In some embodiments, the substrate is a surgical tool. A “surgical tool” as used herein refers to a tool used in a medical procedure that may be inserted into (or touch) the body of a human or animal subject in order to assist or participate in that medical procedure.

“Stability” as used herein in refers to the stability of the drug in a composition in its final product form, whether or not it is deposited on a substrate (e.g., stability of the drug in a coated stent). The term “stability” and/or “stable” in some embodiments is defined by 5% or less degradation of the drug in the final product form. The term stability in some embodiments is defined by 3% or less degradation of the drug in the final product form. The term stability in some embodiments is defined by 2% or less degradation of the drug in the final product form. The term stability in some embodiments is defined by 1% or less degradation of the drug in the final product form.

“Polymer” as used herein, refers to a series of repeating monomeric units that have been cross-linked or polymerized. Any suitable polymer can be used to carry out the present invention. It is possible that the polymers of the invention may also comprise two, three, four or more different polymers. In some embodiments of the invention only one polymer is used. In certain embodiments a combination of two polymers is used. Combinations of polymers can be in varying ratios, to provide compositions with differing properties. Polymers useful in the compositions, devices and methods of the present invention include, for example, stable or inert polymers, organic polymers, organic-inorganic copolymers, inorganic polymers, bioabsorbable, bioresorbable, resorbable, degradable, and biodegradable polymers. Those of skill in the art of polymer chemistry will be familiar with the different properties of polymeric compounds. In some embodiments, the composition comprises a polymer. In some embodiments, the composition comprises an inverse thermosensitive polymer. Such thermosensitive polymers, which are liquid at low temperatures, rapidly transition to gel at physiological temperature. This transition may occur very quickly over approximately one half of a degree Celsius in some embodiments, and this transition temperature can be altered with various compositions, concentrations and buffer solutions. The aqueous, biocompatible polymer is reversible back to a liquid via cooling and is dissolvable (thus such polymers may be referred to as reverse thermosensitive polymer). These polymers are composed of tri-block polymers with two hydrophilic chains connected by a hydrophobic chain. The rapid viscosity transition occurs in response to heat which causes the polymer chains to deform and the hydrophilic arms to align. This leads to the formation of micelles and a subsequent phase change to a viscous gel. The resulting gel is dissolvable and is also reversible back to a liquid with cooling.

The polymers used herein in some embodiments have unique surfactant abilities and extremely low toxicity and immunogenic responses. They may have low acute oral and dermal toxicity and low potential for causing irritation or sensitization, and the general chronic and sub-chronic toxicity is low. They have been found to enhance the therapeutic effect of drugs, and the gene transfer efficiency mediated by adenovirus. Some of these polymers have been considered for various cardiovascular applications, as well as in sickle cell anemia.

In some embodiments, the polymer comprises poloxamer. Several members of this class of polymer, poloxamer 188, poloxamer 407, poloxamer 338, poloxamines 1107 and 1307 show inverse thermosensitivity within the physiological temperature range. In other words, these polymers are members of a class that are soluble in aqueous solutions at low temperature, but gel at higher temperatures. Poloxamer 407 is a biocompatible polyoxypropylene-polyoxyethylene block copolymer having an average molecular weight of about 12,500 and a polyoxypropylene fraction of about 30%; poloxamer 188 has an average molecular weight of about 8400 and a polyoxypropylene fraction of about 20%; poloxamer 338 has an average molecular weight of about 14,600 and a polyoxypropylene fraction of about 20%; poloxamine 1,107 has an average molecular weight of about 14,000, poloxamine 1307 has an average molecular weight of about 18,000. Polymers of this type are also referred to as reversibly gelling because their viscosity increases and decreases with an increase and decrease in temperature, respectively. Such reversibly gelling systems are useful wherever it is desirable to handle a material in a fluid state, but performance is preferably in a gelled or more viscous state. In some embodiments, the polymer comprises poloxamines, e.g., poloxamine 1307 and 1107, which also display inverse thermosensitivity. Certain embodiments comprise poly(ethyleneoxide)/poly(propyleneoxide) block copolymers which have reversible gelling properties have these properties such as Pluronic® poloxamers and Tetronic® poloxamines (BASF, Ludwigshafen, Germany) and generically known as poloxamers and poloxamines, respectively. See U.S. Pat. Nos. 4,188,373, 4,478,822 and U.S. Pat. No. 4,474,751.

The average molecular weights of the poloxamers used in certain embodiments range from about 1,000 to greater than 16,000 daltons. Because poloxamers noted herein may be products of a sequential series of reactions, the molecular weights of these individual poloxamer molecules form a statistical distribution about the average molecular weight. In addition, commercially available poloxamers contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15 to about 50% of the total mass of the polymer.

The inverse thermosensitive polymer used in some embodiments can include a therapeutic agent such as anti-angiogenic agents, hormones, anesthetics, antimicrobial agents (antibacterial, antifungal, antiviral), anti-inflammatory agents, diagnostic agents, or wound healing agents. Similarly, low concentrations of dye (such as methylene blue) or fillers can be added to the inverse thermosensitive polymer.

In some embodiments, the active agent comprises a polymer. In some embodiments, the polymer comprises at least one of polyalkyl methacrylates, polyalkylene-co-vinyl acetates, polyalkylenes, polyurethanes, polyanhydrides, aliphatic polycarbonates, polyhydroxyalkanoates, silicone containing polymers, polyalkyl siloxanes, aliphatic polyesters, polyglycolides, polylactides, polylactide-co-glycolides, poly(e-caprolactone)s, polytetrahalooalkylenes, polystyrenes, poly(phosphasones), copolymers thereof, and combinations thereof.

In embodiments, the polymer is capable of becoming soft after implantation, for example, due to hydration, degradation or by a combination of hydration and degradation. In embodiments, the polymer is adapted to transfer, free, and/or dissociate from a delivery device when at the intervention site due to hydrolysis of the polymer. In various embodiments, the composition comprises a bioabsorbable polymer that is capable of resorbtion in at least one of about 1 day, about 3 days, about 5 days, about 7 days, about 14 days, about 3 weeks, about 4 weeks, about 45 days, about 60 days, about 90 days, about 180 days, about 6 months, about 9 months, about 1 year, about 1 to about 2 days, about 1 to about 5 days, about 1 to about 2 weeks, about 2 to about 4 weeks, about 45 to about 60 days, about 45 to about 90 days, about 30 to about 90 days, about 60 to about 90 days, about 90 to about 180 days, about 60 to about 180 days, about 180 to about 365 days, about 6 months to about 9 months, about 9 months to about 12 months, about 9 months to about 15 months, and about 1 year to about 2 years.

Examples of polymers that may be used in the present invention include, but are not limited to polycarboxylic acids, cellulosic polymers, proteins, polypeptides, polyvinylpyrrolidone, maleic anhydride polymers, polyamides, polyvinyl alcohols, polyethylene oxides, glycosaminoglycans, polysaccharides, polyesters, aliphatic polyesters, polyurethanes, polystyrenes, copolymers, silicones, silicone containing polymers, polyalkyl siloxanes, polyorthoesters, polyanhydrides, copolymers of vinyl monomers, polycarbonates, polyethylenes, polypropylenes, polylactic acids, polylactides, polyglycolic acids, polyglycolides, polylactide-co-glycolides, polycaprolactones, poly(e-caprolactone)s, polyhydroxybutyrate valerates, polyacrylamides, polyethers, polyurethane dispersions, polyacrylates, acrylic latex dispersions, polyacrylic acid, polyalkyl methacrylates, polyalkylene-co-vinyl acetates, polyalkylenes, aliphatic polycarbonates polyhydroxyalkanoates, polytetrahalooalkylenes, poly(phosphasones), polytetrahalooalkylenes, poly(phosphasones), and mixtures, combinations, and copolymers thereof.

The polymers of the present invention may be natural or synthetic in origin, including gelatin, chitosan, dextrin, cyclodextrin, Poly(urethanes), Poly(siloxanes) or silicones, Poly(acrylates) such as [rho]oly(methyl methacrylate), poly(butyl methacrylate), and Poly(2-hydroxy ethyl methacrylate), Poly(vinyl alcohol) Poly(olefins) such as poly(ethylene), [rho]oly(isoprene), halogenated polymers such as Poly(tetrafluoroethylene)—and derivatives and copolymers such as those commonly sold as Teflon® products, Poly(vinylidine fluoride), Poly(vinyl acetate), Poly(vinyl pyrrolidone), Poly(acrylic acid), Polyacrylamide, Poly(ethylene-co-vinyl acetate), Poly(ethylene glycol), Poly(propylene glycol), Poly(methacrylic acid); etc.

Suitable polymers also include absorbable and/or resorbable polymers including the following, combinations, copolymers and derivatives of the following: Polylactides (PLA), Polyglycolides (PGA), PolyLactide-co-glycolides (PLGA), Polyanhydrides, Polyorthoesters, Poly(N-(2-hydroxypropyl) methacrylamide), Poly(1-aspartamide), including the derivatives DLPLA—poly(dl-lactide); LPLA—poly(1-lactide); PDO—poly(dioxanone); PGA-TMC—poly(glycolide-co-trimethylene carbonate); PGA-LPLA—poly(1-lactide-co-glycolide); PGA-DLPLA—poly(dl-lactide-co-glycolide); LPLA-DLPLA—poly(1-lactide-co-dl-lactide); and PDO-PGA-TMC—poly(glycolide-co-trimethylene carbonate-co-dioxanone), and combinations thereof.

“Copolymer” as used herein refers to a polymer being composed of two or more different monomers. A copolymer may also and/or alternatively refer to random, block, graft, copolymers known to those of skill in the art.

As used herein a “gel” may refer to a composition having a viscosity such that the composition remains static, i.e. does not migrate or does not move once deployed in the body. Such a composition may remain in the location of application +/− one distance equal to the size of the implant for at least 1 minute, at least 1 hour, at least 1 day, at least 1 month, at least 6 months, at least about 1 minute, at least about 1 hour, at least about 1 day, at least about 1 month, or at least about 6 months. In some embodiments, a “gel” is a colloid in which the solid disperse phase forms a network in combination with the fluid continuous phase, resulting in a viscous semirigid sol. In some embodiments, a “gel” is a colloid in a more solid form than a sol. In some embodiments, a “gel” is a sol in which the solid particles are meshed such that a rigid or semi-rigid mixture results. In some embodiments, a “gel” is a solid, jelly-like material that can have properties ranging from soft and weak to hard and tough. Gels may be defined as a substantially dilute cross-linked system, which exhibits no flow when in the steady-state. By weight, gels may be mostly liquid, yet they behave like solids due to a three-dimensional cross-linked network within the liquid. It is the crosslinks within the fluid that give a gel its structure (hardness) and contribute to stickiness (tack). In this way gels may be a dispersion of molecules of a liquid within a solid in which the solid is the continuous phase and the liquid is the discontinuous phase.

In some embodiments, the gel may be a hydrogel. Hydrogel (also called aquagel) is a network of polymer chains that are hydrophilic, sometimes found as a colloidal gel in which water is the dispersion medium. Hydrogels are highly absorbent (they can contain over 99.9% water) natural or synthetic polymers. Hydrogels also possess a degree of flexibility very similar to natural tissue, due to their significant water content.

Gels typically comprise a solid three-dimensional network that spans the volume of a liquid medium and ensnares it through surface tension effects. This internal network structure may result from physical bonds (physical gels) or chemical bonds (chemical gels), as well as crystallites or other junctions that remain intact within the extending fluid. Virtually any fluid can be used as an extender including water (hydrogels), oil, and air (aerogel). Both by weight and volume, gels are mostly fluid in composition and thus exhibit densities similar to those of their constituent liquids.

In some embodiments, the gel may be a organogel. An organogel is typically a non-crystalline, non-glassy thermoreversible (thermoplastic) solid material composed of a liquid organic phase entrapped in a three-dimensionally cross-linked network. As used herein, such a gel could comprise a pharmaceutical agent in crystalline form or The liquid can be, for example, an organic solvent, mineral oil, or vegetable oil. The solubility and particle dimensions of the structurant are important characteristics for the elastic properties and firmness of the organogel. Often, these systems are based on self-assembly of the structurant molecules.

In some embodiments, the gel may be a xerogel. A xerogel is a solid formed from a gel by drying with unhindered shrinkage. Xerogels usually retain high porosity (15-50%) and enormous surface area (150-900 m2/g), along with very small pore size (1-10 nm). When solvent removal occurs under hypercritical (supercritical) conditions, the network does not shrink and a highly porous, low-density material known as an aerogel is produced. Heat treatment of a xerogel at elevated temperature produces viscous sintering (shrinkage of the xerogel due to a small amount of viscous flow) and effectively transforms the porous gel into a dense glass.

A “sol” is a type of colloid in which solid particles are suspended in a liquid. A “sol” may be also or alternatively defined as a colloidal suspension of very small solid particles in a continuous liquid medium. A “sol” may be also or alternatively defined as a stable dispersion of either solids in liquids or solids in solids.

In some embodiments, a “colloid” refers to a type of homogeneous mixture in which the dispersed particles do not settle out. A “colloid” may be also or alternatively defined as a substance microscopically dispersed evenly throughout another substance. A colloidal system has two separate phases: a dispersed phase (or internal phase) and a continuous phase (or dispersion medium) in which the colloid is dispersed. A colloidal system may be solid, liquid, or gas.

“Biocompatible” as used herein, refers to any material that does not cause injury or death to the animal or induce a serious adverse reaction in an animal when placed in intimate contact with the animal's tissues. Some relatively benign reaction such as acute inflammation and fibrotic encapsulation occur as part of any normal response to the presence of a foreign substance in contact with tissue. Serious adverse reactions include for example chronic inflammation, infection, excessive fibrotic tissue formation, necrotic cell death, or thrombosis. The terms “biocompatible” and “biocompatibility” when used herein are art-recognized and mean that the referent is neither itself toxic to a host (e.g., an animal or human), nor degrades (if it degrades) at a rate that produces byproducts (e.g., monomeric or oligomeric subunits or other byproducts) at toxic concentrations, causes chronic inflammation or irritation, or induces an immune reaction in the host. It is not necessary that any subject composition have a purity of 100% to be deemed biocompatible. Hence, a subject composition may comprise 99%, 98%, 97%, 96%, 95%, 90% 85%, 80%, 75% or even less of biocompatible agents, e.g., including polymers and other materials and excipients described herein, and still be biocompatible. “Non-biocompatible” as used herein, refers to any material that may cause injury or death to the animal or induce an adverse reaction in the animal when placed in intimate contact with the animal's tissues. Such adverse reactions are as noted above, for example.

The terms “bioabsorbable,” “biodegradable,” “bioerodible,” “bioresorbable,” and “resorbable” are art-recognized synonyms. These terms are used herein interchangeably. Bioabsorbable polymers typically differ from non-bioabsorbable polymers in that the former may be absorbed (e.g. degraded) during use. In certain embodiments, such use involves in vivo use, such as in vivo therapy, and in other certain embodiments, such use involves in vitro use. In general, degradation attributable to biodegradability involves the degradation of a bioabsorbable polymer into its component subunits, or digestion, e.g., by a biochemical process, of the polymer into smaller, polymeric or non-polymeric subunits. In certain embodiments, biodegradation may occur by enzymatic mediation, degradation in the presence of water (hydrolysis) and/or other chemical species in the body, or both. The bioabsorbability of a polymer may be shown in-vitro as described herein or by methods known to one of skill in the art. An in-vitro test for bioabsorbability of a polymer does not require living cells or other biologic materials to show bioabsorption properties (e.g. degradation, digestion). Thus, resorbtion, resorption, absorption, absorbtion, erosion may also be used synonymously with the terms “bioabsorbable,” “biodegradable,” “bioerodible,” and “bioresorbable.” Mechanisms of degradation of a bioabsorbable polymer may include, but are not limited to, bulk degradation, surface erosion, and combinations thereof.

As used herein, the term “biodegradation” encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the bio stability, the biodegradation of any bioabsorbable polymer is usually slower.

“Therapeutically desirable morphology” as used herein refers to the gross form and structure of the pharmaceutical agent, once included in the composition, so as to provide for optimal conditions of ex vivo storage, in vivo preservation and/or in vivo release. Such optimal conditions may include, but are not limited to increased shelf life (i.e., shelf stability), increased in vivo stability, good biocompatibility, good bioavailability or modified release rates. Typically, for the present invention, the desired morphology of a pharmaceutical agent would be crystalline or semi-crystalline or amorphous, although this may vary widely depending on many factors including, but not limited to, the nature of the pharmaceutical agent, the disease to be treated/prevented, the intended storage conditions for the composition prior to use or the location within the body of the interventional site. Preferably at least 10%, 20%, 30%, 40%, 50%, 60%, 70%, 80%, 90%, 95%, 97%, 98%, 99%, 99.5%, and/or 100% of the pharmaceutical agent is in crystalline or semi-crystalline form.

In some embodiments of the methods, compositions and/or devices provided herein, the macrolide immunosuppressive drug is at least 50% crystalline. In some embodiments, the macrolide immunosuppressive drug is at least 75% crystalline. In some embodiments, the macrolide immunosuppressive drug is at least 90% crystalline. In some embodiments of the compositions, methods and/or devices provided herein the macrolide immunosuppressive drug is at least 95% crystalline. In some embodiments of the compositions, methods and/or devices provided herein the macrolide immunosuppressive drug is at least 97% crystalline. In some embodiments of the compositions, methods and/or devices provided herein macrolide immunosuppressive drug is at least 98% crystalline. In some embodiments of the compositions, methods and/or devices provided herein the macrolide immunosuppressive drug is at least 99% crystalline.

In some embodiments of the compositions, methods and/or devices provided herein wherein the pharmaceutical agent is at least 50% crystalline. In some embodiments of the compositions, methods and/or devices provided herein the pharmaceutical agent is at least 75% crystalline. In some embodiments of the compositions, methods and/or devices provided herein the pharmaceutical agent is at least 90% crystalline. In some embodiments of the compositions, methods and/or devices provided herein the pharmaceutical agent is at least 95% crystalline. In some embodiments of the compositions, methods and/or devices provided herein the pharmaceutical agent is at least 97% crystalline. In some embodiments of the compositions, methods and/or devices provided herein pharmaceutical agent is at least 98% crystalline. In some embodiments of the compositions, methods and/or devices provided herein the pharmaceutical agent is at least 99% crystalline.

“Stabilizing agent” as used herein refers to any substance that maintains or enhances the stability of the biological agent. Ideally these stabilizing agents are classified as Generally Regarded As Safe (GRAS) materials by the US Food and Drug Administration (FDA). Examples of stabilizing agents include, but are not limited to carrier proteins, such as albumin, gelatin, metals or inorganic salts. Pharmaceutically acceptable excipients that may be present can further be found in the relevant literature, for example in the Handbook of Pharmaceutical Additives: An International Guide to More Than 6000 Products by Trade Name, Chemical, Function, and Manufacturer; Michael and Irene Ash (Eds.); Gower Publishing Ltd.; Aldershot, Hampshire, England, 1995.

“Intervention site” as used herein refers to the location in the body where the composition is intended to be delivered (by transfer from, freeing from, and/or dissociating from a delivery device). The intervention site can be any substance in the medium surrounding the delivery device, e.g., tissue, cartilage, a body fluid, etc. The intervention site can be the same as the treatment site, i.e., the substance to which the composition is delivered is the same tissue that requires treatment. Alternatively, the intervention site can be separate from the treatment site, requiring subsequent diffusion or transport of the pharmaceutical or other agent away from the intervention site.

“Compressed fluid” as used herein refers to a fluid of appreciable density (e.g., >0.2 g/cc) that is a gas at standard temperature and pressure. “Supercritical fluid,” “near-critical fluid,” “near-supercritical fluid,” “critical fluid,” “densified fluid,” or “densified gas,” as used herein refers to a compressed fluid under conditions wherein the temperature is at least 80% of the critical temperature of the fluid and the pressure is at least 50% of the critical pressure of the fluid, and/or a density of +50% of the critical density of the fluid.

Examples of substances that demonstrate supercritical or near critical behavior suitable for the present invention include, but are not limited to carbon dioxide, isobutylene, ammonia, water, methanol, ethanol, ethane, propane, butane, pentane, dimethyl ether, xenon, sulfur hexafluoride, halogenated and partially halogenated materials such as chlorofluorocarbons, hydrochlorofluorocarbons, hydro fluorocarbons, perfluorocarbons (such as perfluoromethane and perfluoropropane, chloroform, trichloro-fluoromethane, dichloro-difluoromethane, dichloro-tetrafluoroethane) and mixtures thereof. Preferably, the supercritical fluid is hexafluoropropane (FC-236EA), or 1,1,1,2,3,3-hexafluoropropane. Preferably, the supercritical fluid is hexafluoropropane (FC-236EA), or 1,1,1,2,3,3-hexafluoropropane for use in PLGA polymer compositions.

“Sintering” as used herein refers to the process by which parts of the polymer or the entire polymer becomes continuous (e.g., formation of a continuous polymer film). As discussed herein, the sintering process is controlled to produce a fully conformal continuous polymer (complete sintering) or to produce regions or domains of continuous composition while producing voids (discontinuities) in the polymer. As well, the sintering process is controlled such that some phase separation is obtained or maintained between different polymers (e.g., polymers A and B) and/or to produce phase separation between discrete polymer particles. Through the sintering process, the adhesive properties of the composition are improved to reduce flaking of detachment of the composition during manipulation in use. As described herein, in some embodiments, the sintering process is controlled to provide incomplete sintering of the polymer. In embodiments involving incomplete sintering, a polymer is formed with continuous domains, and voids, gaps, cavities, pores, channels or, interstices that provide space for sequestering a therapeutic agent which is released under controlled conditions. Depending on the nature of the polymer, the size of polymer particles and/or other polymer properties, a compressed gas, a densified gas, a near critical fluid or a super-critical fluid may be employed. In one example, carbon dioxide is used to prepare a composition comprising a polymer and a drug, using dry powder and RESS (described below) electrostatic composition processes. In another example, isobutylene is employed in the sintering process. In other examples a mixture of carbon dioxide and isobutylene is employed. In another example, 1,1,2,3,3-hexafluoropropane is employed in the sintering process.

When an amorphous material is heated to a temperature above its glass transition temperature, or when a crystalline material is heated to a temperature above a phase transition temperature, the molecules comprising the material are more mobile, which in turn means that they are more active and thus more prone to reactions such as oxidation. However, when an amorphous material is maintained at a temperature below its glass transition temperature, its molecules are substantially immobilized and thus less prone to reactions. Likewise, when a crystalline material is maintained at a temperature below its phase transition temperature, its molecules are substantially immobilized and thus less prone to reactions. Accordingly, processing drug components at mild conditions, such as the deposition and sintering conditions described herein, minimizes cross-reactions and degradation of the drug component. One type of reaction that is minimized by the processes of the invention relates to the ability to avoid conventional solvents which in turn minimizes -oxidation of drug, whether in amorphous, semi-crystalline, or crystalline form, by reducing exposure thereof to free radicals, residual solvents, protic materials, polar-protic materials, oxidation initiators, and autoxidation initiators.

“Rapid Expansion of Supercritical Solutions” or “RESS” as used herein involves the dissolution of a polymer into a compressed fluid, typically a supercritical fluid, followed by rapid expansion into a chamber at lower pressure, typically near atmospheric conditions. The rapid expansion of the supercritical fluid solution through a small opening, with its accompanying decrease in density, reduces the dissolution capacity of the fluid and results in the nucleation and growth of polymer particles. The atmosphere of the chamber is maintained in an electrically neutral state by maintaining an isolating “cloud” of gas in the chamber. Carbon dioxide, nitrogen, argon, helium, or other appropriate gas is employed to prevent electrical charge transfer from the substrate to the surrounding environment.

“Electrostatic Rapid Expansion of Supercritical Solutions” or “e-RESS” or “eRESS” as used herein refers to Electrostatic Capture as described herein combined with Rapid Expansion of Supercritical Solutions as described herein. In some embodiments, Electrostatic Rapid Expansion of Supercritical Solutions refers to Electrostatic capture as described in the art, e.g., in U.S. Pat. No. 6,756,084, “Electrostatic deposition of particles generated from rapid expansion of supercritical fluid solutions,” incorporated herein by reference in its entirety.

“Solution Enhanced Dispersion of Supercritical Solutions” or “SEDS” as used herein involves a spray process for the generation of polymer particles, which are formed when a compressed fluid (e.g. supercritical fluid, preferably supercritical CO₂) is used as a diluent to a vehicle in which a polymer is dissolved (one that can dissolve both the polymer and the compressed fluid). The mixing of the compressed fluid diluent with the polymer-containing solution may be achieved by encounter of a first stream containing the polymer solution and a second stream containing the diluent compressed fluid, for example, within one spray nozzle or by the use of multiple spray nozzles. The solvent in the polymer solution may be one compound or a mixture of two or more ingredients and may be or comprise an alcohol (including diols, triols, etc.), ether, amine, ketone, carbonate, or alkanes, or hydrocarbon (aliphatic or aromatic) or may be a mixture of compounds, such as mixtures of alkanes, or mixtures of one or more alkanes in combination with additional compounds such as one or more alcohols, (e.g., from 0 or 0.1 to 5% of a Ci to Ci₅ alcohol, including diols, triols, etc.). See for example U.S. Pat. No. 6,669,785, incorporated herein by reference in its entirety. The solvent may optionally contain a surfactant, as also described in, e.g., U.S. Pat. No. 6,669,785.

In one embodiment of the SEDS process, a first stream of fluid comprising a polymer dissolved in a common solvent is co-sprayed with a second stream of compressed fluid. Polymer particles are produced as the second stream acts as a diluent that weakens the solvent in the polymer solution of the first stream. The now combined streams of fluid, along with the polymer particles, flow out of the nozzle assembly into a collection vessel. Control of particle size, particle size distribution, and morphology is achieved by tailoring the following process variables: temperature, pressure, solvent composition of the first stream, flow-rate of the first stream, flow-rate of the second stream, composition of the second stream (where soluble additives may be added to the compressed gas), and conditions of the capture vessel. Typically the capture vessel contains a fluid phase that is at least five to ten times (5-10×) atmospheric pressure.

“Electrostatic Dry Powder Composition” or “e-DPC” or “eDPC” as used herein refers to Electrostatic Capture as described herein combined with Dry Powder Composition. e-DPC deposits material (including, for example, polymer or impermeable dispersed solid) on a substrate as dry powder, using electrostatic capture to attract the powder particles to the substrate. Dry powder spraying (“Dry Powder Composition” or “DPC”) is well known in the art, and dry powder spraying coupled with electrostatic capture has been described, for example in U.S. Pat. Nos. 5,470,603, 6,319,541, and U.S. Pat. No. 6,372,246, all incorporated herein by reference in their entirety. Methods for depositing compositions are described, e.g., in WO 2008/148013, “Polymer Films for Medical Device Composition,” incorporated herein by reference in its entirety.

“Bulk properties” properties of a composition including a pharmaceutical or a biological agent that can be enhanced through the methods of the invention include for example: adhesion, smoothness, conformality, thickness, and compositional mixing.

“Electrostatically charged” or “electrical potential” or “electrostatic capture” as used herein refers to the collection of the spray-produced particles upon a substrate that has a different electrostatic potential than the sprayed particles. Thus, the substrate is at an attractive electronic potential with respect to the particles exiting, which results in the capture of the particles upon the substrate. i.e. the substrate and particles are oppositely charged, and the particles transport through the gaseous medium of the capture vessel onto the surface of the substrate is enhanced via electrostatic attraction. This may be achieved by charging the particles and grounding the substrate or conversely charging the substrate and grounding the particles, by charging the particles at one potential (e.g. negative charge) and charging the substrate at an opposite potential (e.g. positive charge), or by some other process, which would be easily envisaged by one of skill in the art of electrostatic capture.

“Depositing the active agent by an e-RESS, an e-SEDS, or an e-DPC process without electrically charging the substrate” as used herein refers to any of these processes as performed without intentionally electrically charging the substrate. It is understood that the substrate might become electrically charged unintentionally during any of these processes.

“Depositing the active agent by an e-RESS, an e-SEDS, or an e-DPC process without creating an electrical potential between the substrate and a composition apparatus” as used herein refers to any of these processes as performed without intentionally generating an electrical potential between the substrate and the composition apparatus. It is understood that electrical potential between the substrate and the composition apparatus might be generated unintentionally during any of these processes.

“Intimate mixture” as used herein, refers to two or more materials, compounds, or substances that are uniformly distributed or dispersed together.

“Layer” as used herein refers to a material covering a surface or forming an overlying part or segment. Two different layers may have overlapping portions whereby material from one layer may be in contact with material from another layer. Contact between materials of different layers can be measured by determining a distance between the materials. For example, Raman spectroscopy may be employed in identifying materials from two layers present in close proximity to each other.

While layers defined by uniform thickness and/or regular shape are contemplated herein, several embodiments described herein relate to layers having varying thickness and/or irregular shape. Material of one layer may extend into the space largely occupied by material of another layer. For example, in a composition having three layers formed in sequence as a first polymer layer, a pharmaceutical agent layer and a second polymer layer, material from the second polymer layer which is deposited last in this sequence may extend into the space largely occupied by material of the pharmaceutical agent layer whereby material from the second polymer layer may have contact with material from the pharmaceutical layer. It is also contemplated that material from the second polymer layer may extend through the entire layer largely occupied by pharmaceutical agent and contact material from the first polymer layer.

It should be noted however that contact between material from the second polymer layer (or the first polymer layer) and material from the pharmaceutical agent layer (e.g.; a pharmaceutical agent crystal particle or a portion thereof) does not necessarily imply formation of a mixture between the material from the first or second polymer layers and material from the pharmaceutical agent layer. In some embodiments, a layer may be defined by the physical three-dimensional space occupied by crystalline particles of a pharmaceutical agent (and/or biological agent). It is contemplated that such layer may or may not be continuous as physical space occupied by the crystal particles of pharmaceutical agents may be interrupted, for example, by polymer material from an adjacent polymer layer. An adjacent polymer layer may be a layer that is in physical proximity to be pharmaceutical agent particles in the pharmaceutical agent layer. Similarly, an adjacent layer may be the layer formed in a process step right before or right after the process step in which pharmaceutical agent particles are deposited to form the pharmaceutical agent layer.

As described herein, material deposition and layer formation provided herein are advantageous in that the pharmaceutical agent remains largely in crystalline form during the entire process. While the polymer particles and the pharmaceutical agent particles may be in contact, the layer formation process is controlled to avoid formation of a mixture between the pharmaceutical agent particles the polymer particles during formation of the composition.

In some embodiments, the composition comprises a plurality of layers deposited on the substrate, wherein at least one of the layers comprises the active agent. In some embodiments, at least one of the layers comprises a polymer. In some embodiments, the polymer is bioabsorbable. In some embodiments, the active agent and the polymer are in the same layer, in separate layers, or form overlapping layers. In some embodiments, the plurality of layers comprise five layers deposited as follows: a first polymer layer, a first active agent layer, a second polymer layer, a second active agent layer and a third polymer layer.

In some embodiments of the compositions, methods and/or devices provided herein, the composition comprises a plurality of layers deposited on the substrate, wherein at least one of the layers comprises the active agent. In some embodiments, at least one of the layers comprises a polymer. In some embodiments, the polymer is bioabsorbable. In some embodiments, the active agent and the polymer are in the same layer, in separate layers, or form overlapping layers. In some embodiments, the composition comprises a plurality of layers deposited on the substrate, wherein at least one of the layers comprises the pharmaceutical agent. In some embodiments, the pharmaceutical agent and the polymer are in the same layer, in separate layers, or form overlapping layers. In some embodiments, the plurality of layers comprise five layers deposited as follows: a first polymer layer, a first active agent layer, a second polymer layer, a second active agent layer and a third polymer layer. In some embodiments, the plurality of layers comprise five layers deposited as follows: a first polymer layer, a first pharmaceutical agent layer, a second polymer layer, a second pharmaceutical agent layer and a third polymer layer. In some embodiments, the plurality of layers comprise five layers deposited as follows: a first polymer layer, a first active biological agent layer, a second polymer layer, a second active biological agent layer and a third polymer layer.

“Laminate composition” as used herein refers to a composition made up of two or more layers of material. Means for creating a laminate composition as described herein (e.g. a laminate composition comprising bioabsorbable polymer(s) and pharmaceutical agent) may include composition with drug and polymer as described herein (e-RESS, e-DPC, compressed-gas sintering). The process comprises performing multiple and sequential composition preparation steps (with sintering steps for polymer materials) wherein different materials may be deposited in each step, thus creating a laminated structure with a multitude of layers (at least 2 layers) including polymer layers and pharmaceutical agent layers to build the final composition.

“Substantially all of the composition” as used herein refers to at least about 50%, at least about 75%, at least about 85%, at least about 90%, at least about 95%, at least about 97%, and/or at least about 99% percent of the composition that was present prior to use.

“Delivering at least a portion” as used herein in the context of a composition and/or active agent at an intervention site refers to an amount and/or percentage of a composition and/or active agent that is delivered to an intervention site. In some embodiments, at least about 10%, at least about 20%, at least about 30%, at least about 50%, at least about 75%, at least about 85%, at least about 90%, at least about 95%, and/or at least about 99% of the composition and/or active agent is delivered to the intervention site.

In some embodiments, the composition is adapted to deliver at least about 10%, at least about 20%, at least about 30%, at least about 50%, at least about 75%, at least about 85%, at least about 90%, at least about 95%, and/or at least about 99% of the active agent to the intervention site.

In some embodiments, transferring at least a portion of the active agent comprises transferring at least about 3%, at least about 5%, at least about 10%, at least about 20%, at least about 30%, greater than 35%, at least about 50%, at least about 75%, at least about 85%, at least about 90%, at least about 95%, and/or at least about 99% of the active agent from the composition.

The term “adapted to transfer at least a portion” of the composition or active agent to an intervention site refers to a delivery device that is designed to transfer any portion of the composition or active agent to an intervention site.

The term “adapted to free” a portion of a composition and/or active agent from the substrate refers to a delivery device, composition, and/or substrate that is designed to free a certain percentage of the composition and/or active agent from the substrate.

A linear release may mean, depending on the embodiment, that upon analysis of elution data gathered according to methods noted herein or known to one of skill in the art using a least squares regression analysis of the data that there is an r-squared value of the best fit line that is at least 0.8 during the period noted, for example during any one or more of the following periods: from implantation until 30% of the agent is eluted, 50% of the agent is eluted, from implantation until 65% of the agent is eluted, from implantation until 20% of the agent is eluted, from implantation until 40% of the agent is eluted, from implantation until 45% of the agent is eluted, from implantation until 60% of the agent is eluted, from days 0 to 5, from days 0 to 9, from days 0 to 7, from days 0 to 14, from days 0 to 21, from days 0 to 28, from day 0 to one month, from days 1 to 5, from days 1 to 9, from days 1 to 7, from days 1 to 14, from days 1 to 21, from days 1 to 28, from day 1 to one month, and from day 1 to two months.

A linear release may mean, depending on the embodiment, that upon analysis of elution data gathered according to methods noted herein or known to one of skill in the art using a linear regression analysis of the data that there is an r-squared value of the best fit line that is at least 0.8 during the period noted, for example during any one or more of the following periods: from implantation until 30% of the agent is eluted, 50% of the agent is eluted, from implantation until 65% of the agent is eluted, from implantation until 20% of the agent is eluted, from implantation until 40% of the agent is eluted, from implantation until 45% of the agent is eluted, from implantation until 60% of the agent is eluted, from days 0 to 5, from days 0 to 9, from days 0 to 7, from days 0 to 14, from days 0 to 21, from days 0 to 28, from day 0 to one month, from days 1 to 5, from days 1 to 9, from days 1 to 7, from days 1 to 14, from days 1 to 21, from days 1 to 28, from day 1 to one month, and from day 1 to two months.

A linear release may mean, depending on the embodiment, that upon analysis of elution data gathered according to methods noted herein or known to one of skill in the art using curve-fitting of the data that there is a linear relationship between the time points and the elution data which has an r-squared value of the linear best fit line that is at least 0.8 during the period noted, for example during any one or more of the following periods: from implantation until 30% of the agent is eluted, 50% of the agent is eluted, from implantation until 65% of the agent is eluted, from implantation until 20% of the agent is eluted, from implantation until 40% of the agent is eluted, from implantation until 45% of the agent is eluted, from implantation until 60% of the agent is eluted, from days 0 to 5, from days 0 to 9, from days 0 to 7, from days 0 to 14, from days 0 to 21, from days 0 to 28, from day 0 to one month, from days 1 to 5, from days 1 to 9, from days 1 to 7, from days 1 to 14, from days 1 to 21, from days 1 to 28, from day 1 to one month, and from day 1 to two months.

A linear release may mean, depending on the embodiment, that upon analysis of elution data gathered according to methods noted herein or known to one of skill in the art using a least squares regression analysis of the data that there is an r-squared value of the best fit line that is at least 0.9 during the period noted, for example during any one or more of the following periods: from implantation until 30% of the agent is eluted, 50% of the agent is eluted, from implantation until 65% of the agent is eluted, from implantation until 20% of the agent is eluted, from implantation until 40% of the agent is eluted, from implantation until 45% of the agent is eluted, from implantation until 60% of the agent is eluted, from days 0 to 5, from days 0 to 9, from days 0 to 7, from days 0 to 14, from days 0 to 21, from days 0 to 28, from day 0 to one month, from days 1 to 5, from days 1 to 9, from days 1 to 7, from days 1 to 14, from days 1 to 21, from days 1 to 28, from day 1 to one month, and from day 1 to two months.

A linear release may mean, depending on the embodiment, that upon analysis of elution data gathered according to methods noted herein or known to one of skill in the art using a linear regression analysis of the data that there is an r-squared value of the best fit line that is at least 0.9 during the period noted, for example during any one or more of the following periods: from implantation until 30% of the agent is eluted, 50% of the agent is eluted, from implantation until 65% of the agent is eluted, from implantation until 20% of the agent is eluted, from implantation until 40% of the agent is eluted, from implantation until 45% of the agent is eluted, from implantation until 60% of the agent is eluted, from days 0 to 5, from days 0 to 9, from days 0 to 7, from days 0 to 14, from days 0 to 21, from days 0 to 28, from day 0 to one month, from days 1 to 5, from days 1 to 9, from days 1 to 7, from days 1 to 14, from days 1 to 21, from days 1 to 28, from day 1 to one month, and from day 1 to two months.

A linear release may mean, depending on the embodiment, that upon analysis of elution data gathered according to methods noted herein or known to one of skill in the art using curve-fitting of the data that there is a linear relationship between the time points and the elution data which has an r-squared value of the linear best fit line that is at least 0.9 during the period noted, for example during any one or more of the following periods: from implantation until 30% of the agent is eluted, 50% of the agent is eluted, from implantation until 65% of the agent is eluted, from implantation until 20% of the agent is eluted, from implantation until 40% of the agent is eluted, from implantation until 45% of the agent is eluted, from implantation until 60% of the agent is eluted, from days 0 to 5, from days 0 to 9, from days 0 to 7, from days 0 to 14, from days 0 to 21, from days 0 to 28, from day 0 to one month, from days 1 to 5, from days 1 to 9, from days 1 to 7, from days 1 to 14, from days 1 to 21, from days 1 to 28, from day 1 to one month, and from day 1 to two months.

The term SS refers to the following chemical substituent.

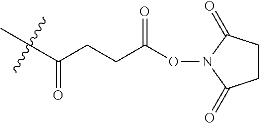 [SMILES: *C(=O)CCC(=O)ON1C(=O)CCC1=O]

The term SG refers to the following chemical substituent.

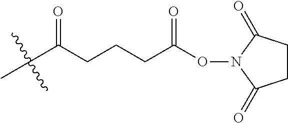 [SMILES: *C(=O)CCCC(=O)ON1C(=O)CCC1=O]

The term SA refers to the following chemical substituent.

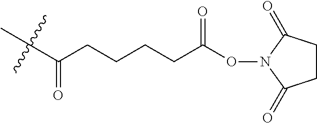 [SMILES: *C(=O)CCCCC(=O)ON1C(=O)CCC1=O]

The term SSub refers to the following chemical substituent.

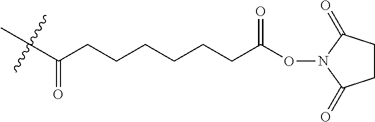 [SMILES: *C(=O)CCCCCCC(=O)ON1C(=O)CCC1=O]

The term SSeb refers to the following chemical substituent.

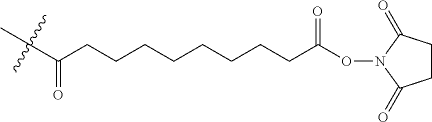 [SMILES: *C(=O)CCCCCCCCC(=O)ON1C(=O)CCC1=O]

### Methods of Manufacturing Generally

In some embodiments, a composition is formed on the substrate by a process comprising depositing a polymer and/or the active agent by an e-RESS, an e-SEDS, or an e-DPC process. In some embodiments, the composition is formed on a substrate by a process comprising depositing the active agent by an e-RESS, an e-SEDS, or an e-DPC process without electrically charging the substrate. In some embodiments, the composition is formed on the substrate by a process comprising depositing the active agent on the substrate by an e-RESS, an e-SEDS, or an e-DPC process without creating an electrical potential between the substrate and a composition apparatus used to deposit the active agent. The composition is then released from the substrate and formed into a dosage form such as a composition suitable for injection; a coating for transdermal delivery device or formed into an oral dosage form.

Means for creating the bioabsorbable polymer(s)+drug (s) composition:
Provide a composition deposition substrate (such as a mandrel) for preparing the composition. The composition is released from the substrate for further processing and or packaging.
Spray-coat the composition-form with drug and polymer as is done in the described process (e-RESS, e-DPC, compressed-gas sintering).
Perform multiple and sequential composition—sintering steps where different materials may be deposited in each step, thus creating a laminated structure with a multitude of thin layers of drug(s), polymer(s) or drug+polymer that build the final composition.
Perform the deposition of polymer(s)+drug(s) laminates with the inclusion of a mask on the composition deposition substrate. Such a mask could be as simple as a non-conductive mandrel inserted through the internal diameter of the composition form. This masking could take place prior to any layers being added, or be purposefully inserted after several layers are deposited continuously around the entire composition-form.
Release the composition from the composition deposition substrate, and
Condition the composition for forming an injectable dosage form, a transdermal dosage form or a dosage form suitable for oral administration.

It is also contemplated that the compositions disclosed herein can be prepared by processing the polymer, the active pharmaceutical ingredient, the core if present and/or the pharmaceutically customary excipients by injection molding, extrusion, wet granulation, casting, spreading, spraying or compression to form compositions having the advantages described herein.

In some embodiments, the composition comprises a microstructure. In some embodiments, particles of the active agent are sequestered or encapsulated within the microstructure. In some embodiments, the microstructure comprises microchannels, micropores and/or microcavities. In some embodiments, the microstructure is selected to allow sustained release of the active agent. In some embodiments, the microstructure is selected to allow controlled release of the active agent.

Another advantage of the present invention is the ability to create a dosage form with a controlled (dialed-in) drug-release profile. Via the ability to have different materials in each layer of the laminate structure and the ability to control the location of drug(s) independently in these layers, the method enables a composition that could release drugs at very specific release profiles, programmed sequential and/or parallel release profiles. Also, the present invention allows controlled release of one drug without affecting the release of a second drug (or different doses of the same drug).

Provided herein is a method of forming a composition, wherein the composition comprises an active agent, the method comprising: providing the substrate; and forming the composition on at least a portion of the substrate by depositing the active agent by on the substrate by at least one of an e-RESS, an e-SEDS, and an e-DPC process, wherein forming the composition results in at least a portion of the composition being adapted to transfer from the substrate to prepare a dosage form containing the composition.

One embodiment provides a drug delivery composition comprising at least one hydrogel and at least one active agent; wherein the active agent is present in crystalline form and is embedded in the hydrogel.

Another embodiment provides the drug delivery composition wherein the composition indicates the presence of said pharmaceutical agent in crystalline form upon analysis by an analytical method selected from: (a) X-ray spectroscopy, (b) scanning electron microscopy (SEM), (c) Raman spectrum, (d) Differential Scanning calorimetry (DSC), (e) Wide Angle X-ray Scattering (WAXS) spectroscopy, and (f) wide angle radiation scattering spectroscopy.

Another embodiment provides the drug delivery composition wherein curing of the hydrogel occurs in-vivo.

Another embodiment provides the drug delivery composition wherein the composition is formed to provide a selected active agent controlled release profile. Another embodiment provides the drug delivery composition wherein the composition is in a form suitable for a mode of administration selected from: (a) injection, (b) intramuscular injection, (c) subcutaneous injection, (d) intrathecal injection, (e) intramuscular injection, (f) intraarticular injection, (g) intraperitoneal injection, (h) dermal administration, and (i) during a surgical procedure.

Another embodiment provides the drug delivery composition wherein the composition is adapted for delivery to at least one of: the front of the eye, the back of the eye, the location of a tumor, the location of cancerous cells, the location of cancerous tissue, the former location of cancerous tissue, the former location of cancerous cells, the former location of a tumor, the brain, a neurologic site, a location where inflammation is occurring, a location where inflammation may occur, a location where inflammation is expected to occur.

Another embodiment provides the drug delivery composition wherein the composition is adapted for dural repair.

Another embodiment provides the drug delivery composition wherein the composition is adapted to treat at least one of: intracranial aneurysms, tumors, and spinal disc disease.

Another embodiment provides the drug delivery composition wherein the composition is adapted to stop, slow, or prevent cervical spinal fluid leaks.

Another embodiment provides the drug delivery composition wherein the composition is capable of maintaining a seal under high pressures.

Another embodiment provides the drug delivery composition wherein the composition degrades as natural healing occurs.

Another embodiment provides the drug delivery composition wherein the composition acts as an adhesion barrier.

Another embodiment provides the drug delivery composition wherein the composition acts as a bandage.

Another embodiment provides the drug delivery composition wherein the composition is adapted to cover an opening in the eye and providing a protective barrier to the portion of the eye that is covered.

Another embodiment provides the drug delivery composition wherein the composition comprises voids.

Another embodiment provides the drug delivery composition wherein tissue ingrowth occurs in the voids as tissue heals.

Another embodiment provides the drug delivery composition wherein the composition is in the form of a mesh.

Another embodiment provides the drug delivery composition wherein the composition is adapted to seal tissue.

Another embodiment provides the drug delivery composition wherein the composition may be used in place of or in combination with tacks, staples, sutures, or O-rings in surgical procedures to seal tissue.

Another embodiment provides the drug delivery composition wherein the composition bioabsorbs or degrades as tissue grows into the voids of the mesh and into areas where the hydrogel has been degraded or absorbed.

Another embodiment provides the drug delivery composition wherein the hydrogel is biodegradable.

Another embodiment provides the drug delivery composition wherein the hydrogel is anti-microbial.

Another embodiment provides the drug delivery composition wherein the biologically active agent is encapsulated in microparticles or nanoparticles.

Another embodiment provides the drug delivery composition wherein the composition further comprises one or more agents that modulate the viscosity of the composition.

Another embodiment provides the drug delivery composition wherein the agent that modulates the viscosity of the composition lowers the viscosity of the composition at room temperature.

Another embodiment provides the drug delivery composition wherein the agent that modulates the viscosity of the composition increases the viscosity of the composition at temperatures above 35° C.

Another embodiment provides the drug delivery composition wherein the active agent is selected from rapamycin, a prodrug, a derivative, an analog, a hydrate, an ester, and a salt thereof.

Another embodiment provides the drug delivery composition wherein the active agent is selected from one or more of sirolimus, everolimus, zotarolimus and biolimus.

Another embodiment provides the drug delivery composition wherein the active agent comprises a macrolide immunosuppressive (limus) drug.

Another embodiment provides the drug delivery composition wherein the active agent is a macrolide immunosuppressive drug selected from one or more of rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

Another embodiment provides the drug delivery composition wherein the active agent is at least 50% crystalline, at least 75% crystalline, or at least 90% crystalline.

Another embodiment provides the drug delivery composition wherein the hydrogel comprises a polymer.

Another embodiment provides the drug delivery composition wherein the polymer comprises a PLGA copolymer.

Another embodiment provides the drug delivery composition wherein the polymer comprises a first PLGA copolymer with a ratio of about 40:60 to about 60:40 and a second PLGA copolymer with a ratio of about 60:40 to about 90:10.

Another embodiment provides the drug delivery composition wherein the polymer is selected from the group PLGA, PGA poly(glycolide), LPLA poly(1-lactide), DLPLA poly(dl-lactide), PCL poly(e-caprolactone) PDO, poly(dioxolane) PGA-TMC, 85/15 DLPLG p(dl-lactide-co-glycolide), 75/25 DLPL, 65/35 DLPLG, 50/50 DLPLG, TMC poly(trimethylcarbonate), p(CPP:SA) poly(1,3-bis-p-(carboxyphenoxy)propane-co-sebacic acid).

Another embodiment provides the drug delivery composition wherein between 25% and 45% of the total amount of active agent in the composition is released after 24 hours in vitro release in a 1:1 spectroscopic grade ethanol/phosphate buffer saline at pH 7.4 and 37° C.; wherein the amount of the active agent released is determined by measuring UV absorption at 278 nm by a diode array spectrometer.

Another embodiment provides the drug delivery composition wherein the composition is suitable for use in a mode of administration selected from: (a) injection into the bladder to treat bladder cancer, (b) injection into the prostate gland to treat prostate cancer, (c) injection into or near the vitreous humor of the eye to treat ocular disease, and (d) injection into the nasal turbinates to treat chronic sinusitis.

Another embodiment provides the drug delivery composition wherein the composition is suitable for injection into an intervention site wherein the intervention site is selected from: (a) the wall of a body cavity, (b) the wall of a body cavity resulting from partial or complete tumor removal, (c) a cannulized site within a subject, (d) a nasal turbinate, (e) within the reproductive system of a subject, (f) within the urinary system of a subject, (g) located at a tumor site, (f) a location in the ear, (g) a location in the esophagus, (h) a location in the larynx, (i) a location of an injury, (j) an infection site, (k) a surgery site, (l) an ocular site, (m) an inflammatory site, or (n) an arthritic joint.

Another embodiment provides the drug delivery composition wherein the composition is adapted to treat an ailment selected from: (a) an ailment of the reproductive system, (b) an ailment of the urinary system.

Another embodiment provides the drug delivery composition wherein the composition is adapted for delivery to at least one of an intervention site, an infection site, and an inflammatory site.

Another embodiment provides the drug delivery composition adapted for delivery to an intervention site wherein the intervention site is located in the urinary system of a subject.

Another embodiment provides the drug delivery composition adapted for delivery to an intervention site wherein the intervention site is located at a tumor site.

Another embodiment provides the drug delivery composition adapted for delivery to a tumor site wherein the tumor site is where a tumor is located.

Another embodiment provides the drug delivery composition adapted for delivery to a tumor site wherein the tumor site is where a tumor was located prior to removal and/or shrinkage of the tumor.

Another embodiment provides the drug delivery composition adapted for delivery to an intervention site wherein the intervention site is located in the ear.

Another embodiment provides the drug delivery composition adapted for delivery to an intervention site wherein the intervention site is located in the esophagus.

Another embodiment provides the drug delivery composition adapted for delivery to an intervention site wherein the intervention site is located in the larynx.

Another embodiment provides the drug delivery composition adapted for delivery to an intervention site wherein the intervention site is a location of an injury.

Another embodiment provides the drug delivery composition adapted for delivery to an intervention site wherein the intervention site is an infection site.

Another embodiment provides the drug delivery composition wherein the composition is adapted for delivery to an inflammatory site wherein the inflammatory site is a site wherein inflammation may occur, and wherein the active agent is capable of substantially preventing the inflammation.

Another embodiment provides the drug delivery composition wherein the composition is adapted for delivery to an inflammatory site wherein the inflammatory site is a site wherein inflammation may occur, and wherein the active agent is capable of substantially reducing the inflammation.

Another embodiment provides the drug delivery composition wherein the composition is adapted for delivery to an infection site wherein the infection site is a site wherein an infection may occur, and wherein the active agent is capable of substantially preventing the infection.

Another embodiment provides the drug delivery composition wherein the composition is adapted for delivery to an infection site wherein the infection site is a site wherein an infection has occurred, and wherein the active agent is capable of slowing spread of the infection.

Another embodiment provides the drug delivery composition wherein the composition is adapted for delivery to an intervention site wherein the intervention site is an ocular site.

Another embodiment provides the drug delivery composition wherein the composition is capable of at least one of: retarding healing, delaying healing, and preventing healing.

Another embodiment provides the drug delivery composition wherein the composition is capable of at least one of: retarding, delaying, and preventing the inflammatory phase of healing.

Another embodiment provides the drug delivery composition wherein the composition is capable of at least one of: retarding, delaying, and preventing the proliferative phase of healing.

Another embodiment provides the drug delivery composition wherein the active agent and the polymer are in the same layer, in separate layers, or form overlapping layers. Another embodiment provides the drug delivery composition wherein the active agent is uniformly dispersed within the composition.

Another embodiment provides the drug delivery composition wherein the active agent release profile is a linear release profile until 30% of the total content of active agent is released.

Another embodiment provides the drug delivery composition wherein the active agent release profile is a linear release profile until 50% of the total content of active agent is released.

Another embodiment provides the drug delivery composition wherein the composition releases the active agent at a selected therapeutic level over a period of at least 48 hours after injection.

Another embodiment provides the drug delivery composition wherein the composition reduces or eliminates adverse toxic effect associated with oral formulation of the active agent.

Another embodiment provides the drug delivery composition wherein the composition is suitable for intramuscular injection or intraperitoneal injection.

Another embodiment provides the drug delivery composition wherein the active agent is delivered transdermally from the composition at a selected active agent release profile.

Another embodiment provides the transdermal drug delivery composition further comprising a microprotrusion member having a plurality of stratum corneum piercing microprotrusions thereon and being adapted for piercing the stratum corneum to improve transdermal flux of the composition.

Another embodiment provides the transdermal drug delivery composition wherein the composition releases the active agent at a selected therapeutic level over a period of at least 48 hours after application of the composition.

Another embodiment provides the transdermal drug delivery composition wherein the composition reduces or eliminates adverse toxic effect associated with oral formulation of the active agent.

Another embodiment provides the drug delivery composition wherein the composition is adapted for delivery intra-articularly.

Another embodiment provides the drug delivery composition wherein the hydrogel comprises a hydrogel composition that is derived from an activated polyalkylene glycol diacid derivative and a crosslinking agent.

Another embodiment provides the drug delivery composition comprising a hydrogel composition wherein the activated polyalkylene diacid derivative is represented by formula (I):

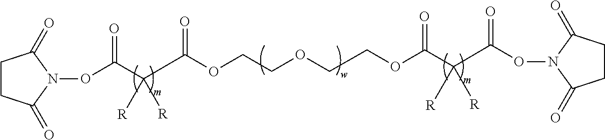

wherein, independently for each occurrence, R is H or lower alkyl; m is 2-20 inclusive; and w is 5 to 1,000 inclusive.

Another embodiment provides the drug delivery composition comprising a hydrogel composition wherein the crosslinking agent is a polyalkyleneimine or trilysine.

Another embodiment provides the drug delivery composition comprising a hydrogel composition wherein the crosslinking agent is polyethyleneimine having a molecular weight of about 2000.

Another embodiment provides the drug delivery composition comprising a hydrogel composition wherein the crosslinking agent is trilysine.

Another embodiment provides the drug delivery composition comprising a hydrogel component of formula (I) wherein m is 2-10 inclusive.

Another embodiment provides the drug delivery composition comprising a hydrogel component of formula (I) wherein m is 2.

Another embodiment provides the drug delivery composition comprising a hydrogel component of formula (I) wherein m is 3.

Another embodiment provides the drug delivery composition comprising a hydrogel component of formula (I) wherein m is 4.

Another embodiment provides the drug delivery composition comprising a hydrogel component of formula (I) wherein m is 6.

Another embodiment provides the drug delivery composition comprising a hydrogel component of formula (I) wherein m is 8.

Another embodiment provides the drug delivery composition comprising a hydrogel component of formula (I) wherein w is 20 to 120 inclusive.

Another embodiment provides the drug delivery composition comprising a hydrogel component of formula (I) wherein w is 120 to 250 inclusive.

Another embodiment provides the drug delivery composition comprising a hydrogel composition wherein the activated polyalkylene glycol diacid derivative is R-PEGₙ-R; wherein n represents the number average molecular weight of the PEG and is about 2000 to about 12,000 inclusive; and R is SS, SG, SA, SSub, or SSeb.

Another embodiment provides the drug delivery composition comprising a hydrogel composition wherein the activated polyalkylene glycol diacid derivative is PEG₃₃₅₀-(SS)₂.

Another embodiment provides the drug delivery composition comprising a hydrogel composition wherein the activated polyalkylene glycol diacid derivative is (SS)-PEG₃₃₅₀-(SS).

Another embodiment provides the drug delivery composition comprising a hydrogel composition wherein the activated polyalkylene glycol diacid derivative is PEG₃₃₅₀-(SG)₂.

Another embodiment provides the drug delivery composition comprising a hydrogel composition wherein the activated polyalkylene glycol diacid derivative is (SG)-PEG₃₃₅₀-(SG).

Another embodiment provides the drug delivery composition comprising a hydrogel composition wherein the activated polyalkylene glycol diacid derivative is PEG₃₃₅₀-(SA)₂.

Another embodiment provides the drug delivery composition comprising a hydrogel composition wherein the activated polyalkylene glycol diacid derivative is (SA)-PEG₃₃₅₀-(SA).

Another embodiment provides the drug delivery composition comprising a hydrogel composition wherein the activated polyalkylene glycol diacid derivative is PEG₃₃₅₀-(SSeb)₂.

Another embodiment provides the drug delivery composition comprising a hydrogel composition wherein the activated polyalkylene glycol diacid derivative is (SSeb)-PEG₃₃₅₀-(SSeb).

Another embodiment provides the drug delivery composition comprising a hydrogel composition wherein the activated polyalkylene glycol diacid derivative is PEG₃₃₅₀-(SSub)₂.

Another embodiment provides the drug delivery composition comprising a hydrogel composition wherein the activated polyalkylene glycol diacid derivative is (SSub)-PEG₃₃₅₀-(SSub).

Another embodiment provides the drug delivery composition comprising a hydrogel component wherein the weight percent crosslinker is between about 5% and about 50%.

Another embodiment provides the drug delivery composition comprising a hydrogel component wherein the weight percent crosslinker is about 15%.

Another embodiment provides the drug delivery composition comprising a hydrogel component wherein the ratio of activated esters on the polyalkylene glycol diacid derivatives to primary amines on the crosslinking agent is in the range from about 0.10:1 to about 10:1.

Another embodiment provides the drug delivery composition comprising a hydrogel component wherein the ratio of activated esters on the polyalkylene glycol diacid derivatives to primary amines on the crosslinking agent is in the range from about 0.75:1 to about 1.3:1.

Another embodiment provides the drug delivery composition comprising a hydrogel component wherein R is methyl or H.

Another embodiment provides the drug delivery composition comprising a hydrogel component wherein R is H.

Rapamycin (also known as Sirolimus) forms a complex with FK Binding Protein-12 (FKBP-12). This complex binds to and inhibits activation of a key regulatory kinase, mammalian Target of Rapamycin (mTOR). This is believed to suppress cytokine-driven cell proliferation, inhibiting cell cycle progression from the G1 to S phase. Rapamycin can be used to treat a wide variety of diseases brought on by uncontrolled cell growth due to the importance of mTOR in regulating cell proliferation in many different types of cells.

The clinical utility of Rapamycin, currently formulated as Rapamune® oral tablets or solution, is limited by its poor aqueous solubility, low bioavailability (<14%), high protein binding and extensive hepatic biotransformation. Once daily oral dosing results in non-steady state pharmacokinetics requiring close monitoring of serum peak and trough levels to maintain drug in a therapeutic range. Depending on the immunosuppressive dose used (2 or 5 mg daily), blood concentrations can range from 6-50 ng/ml. A controlled release formulation that can deliver drug at a constant rate could improve drug effectiveness and reduce the risk of toxicity. Delivery of drug from implanted depots or other types of reservoir technologies could improve bioavailability and provide delivery closer to a selected target.

The following disease targets are known to be susceptible to mTOR inhibitors such as rapamycin. These pathologies might be better treated with a therapeutic modality that provides a more constant inhibition of mTOR activity or an increase in the local concentration of that inhibitor.

### Illustrative Disease Targets:

Plas DR and Thomas G, “Tubers and tumors: rapamycin therapy for benign and malignant tumors”, Curr Opin Cell Bio 21: 230-236, (2009) lists the following targets: Immunosuppression following organ transplantation, prevention of restenosis following cardiovascular intervention, treatment of phakomatosis and harmartomatous diseases and treatment of cancers, particularly those linked to Akt activation such as renal cell carcinoma, glioblastoma and prostate cancer.

### Immunosuppression

The immunosuppressive activity of rapamycin is due, in part, from its ability to inhibit the cytokine mediated proliferation of T- and B-lymphocytes, both of which play major roles in graft rejection. In fact, rapamycin has been shown to affect the expression of hundreds of genes in these lymphocytes. Activation of lymphocytes and transplant allograft rejection occurs both as a systemic and local response to the graft. Local delivery of immunosuppressive therapy in the early transplantation period directly into the graft is, therefore, a possible way to prevent rejection and reduce the systemic dose and the accompanying systemic adverse effects of these drugs.

Preclinical evaluation of two-week continuous intraportal administration of immunosuppressant therapy was evaluated in a model of canine liver transplantation. The result was increased levels of therapeutic agent in the graft and dramatically increased survival compared to animals given comparable amounts of systemic therapy.

Another example of where local immunosuppression provides clear benefit is with corneal transplantation. There is a “high risk” patient population in which up to 60% of corneal allografts are rejected despite the use of topical steroids. The failure in suppression of graft rejection usually results from the inability to achieve effective drug concentrations in the cornea and anterior chamber. Recently a novel implant technology using nanoparticles loaded with rapamycin has been shown to demonstrate improved graft survival in a rabbit model of corneal transplantation.

While the currently available dosage forms of rapamycin reduce the risk of transplant rejection, it is clear from these examples that sustained release of drug into or near the graft might provide additional benefit, particularly in the first few weeks after implant. Continuous local infusion of rapamycin through an extracorporeal catheter might increase the risk of infection or other morbidity. The technology disclosed herein offers a potentially safer solution with the local implantation of biodegradable drug depot capable of sustaining release of active drug at a constant rate for a period of approximately two weeks.

### Tuberous Sclerosis

Tuberous sclerosis is an autosomal dominant, multi-system tumor disorder affecting brain, kidneys, lungs, heart and skin. The disease occurs as the result of a mutation in either the TSC1 or the TSC2 genes that, under normal circumstances, produce proteins that bind to form a tumor suppressor complex. Failure of the tumor suppressor complex to function results in constitutively active mTOR. Inhibitors of mTOR such as rapamycin are used, therefore to treat tuberous sclerosis. Systemic rapamycin therapy reduces tumors but the response is incomplete, often temporary and associated with significant, if tolerable, side effects. More effective treatment strategies are sought.

One strategy for improved treatment is the local application of rapamycin to tumor sites to increase the concentration of mTOR inhibitor within the tumor. Topical delivery of rapamycin by application of a patch near the skin lesions resulted in reduced tumor growth in a mouse model of tuberous sclerosis. The technology disclosed herein could be used to generate dermal drug delivery patches with the capacity to deliver active rapamycin for extended periods.

### Other Cancers

Rapamycin's target, mTOR, plays an important role in the proliferation of numerous cell types including many cancer cell types. As is the case with immunosuppression, improved effectiveness and reduced toxicity could be achieved with a drug delivery technology capable of sustained, controlled release of drug that would maintain optimal therapeutic serum and tumor levels of anti-proliferative drug. Specifically, with regard to adenocarcinomas it is known that continuous infusion of rapamycin results in significantly greater tumor inhibition than daily administration. Although rapamycin and other mTOR inhibitors have the potential to greatly impact tumor growth, progression and resistance, they have enjoyed only modest success in many clinical trials. This may be due to less than optimal drug dosing or the activity of negative feedback loops that limit drug effect.

To provide additional and/or improved anti-cancer therapy, the technology disclosed herein could be used to generate a rapamycin-delivery implant capable of providing sustained release of active drug either systemically in the case of metastatic disease or more directly into a solid tumor. In addition, since mTOR dysregulation is known to be an important element of a number of different brain tumors such as neurofibromatosis and the brain tumors from tuberous sclerosis, a form of rapamycin delivery that could be implanted beyond the blood brain barrier is potentially advantageous.

### Anti-TNFα

One of rapamycin's myriad of cellular effects is the inhibition of Tumor Necrosis Factor-α (TNFα) release from vascular smooth muscle cells. TNFα is a proinflammatory, prothrombotic cytokine elevated in response to endogenous disease such as arthritis and atherosclerosis and also released in response to vascular injury, particularly in response to a foreign body. Part of the rapamycin-eluting stent's antirestenotic effectiveness may be due to its ability to inhibit TNFα release. Other types of medical implants also cause release of TNFα. The release of this cytokine can aggravate on-going disease and also result in damage to the implant itself. For example, in the case of joint replacements such as hip or knee arthroplasty, wear debris from the prosthesis can initiate an inflammatory response including release of TNFα which in turn leads to recruitment of activated osteoclasts that can degrade the bone-implant interface.

A novel solution to this issue would be a surface coating on the prosthetic that released rapamycin as an inhibitor of TNFα (in addition to its other anti-inflammatory effects). The composition formation technology disclosed herein could lend itself well to this type of application.

### Autism and Alzheimer's Disease

Cellular events such as phosphorylation of neural proteins are partially responsible for the processing of neural input and the generation of memories. The kinase, mTOR, acts as a “node of convergence” in which many different types of signals are translated into phosphorylation events. Although disorders like autism can arise from multiple genetic alterations, 5-10% of these directly involve mTOR signaling or translational control. mTOR dysregulation is also a characteristic of Alzheimer's disease.

Although rapamycin is a potent means to inhibit mTOR, its use to treat these complex diseases is still highly experimental. mTOR is not a simple “on/off” switch and both too much or too little mTOR activity can negatively impact neuronal signaling. In addition, there is evidence that even within a single cell proper functioning of mTOR may involve increased activity in one compartment and decreased activity in another. While intriguing, much more will have to be learned before undertaking the optimized delivery of rapamycin to treat these types of diseases.

The methods disclosed herein can produce drug delivery products comprised of stable, concentrated rapamycin that elutes from a biodegradable matrix at a constant rate for a prolonged period of time. Drug delivery kinetics can be varied based on critical formulation parameters. The use of these types of compositions can potentially provide both novel and better optimized therapeutic options to treat a wide variety of disease states.

One embodiment provides a drug delivery composition comprising at least one polymer and at least one active agent; wherein the active agent is present in crystalline form on at least one region of an outer surface of the composition and wherein active agent surface content is adjusted to provide a selected active agent release profile.

One embodiment provides a drug delivery composition wherein presence of active agent on at least a region of the surface of the composition is determined by cluster secondary ion mass spectrometry (cluster SIMS).

One embodiment provides a drug delivery composition wherein presence of active agent on at least a region of the surface of the composition is determined by generating cluster secondary ion mass spectrometry (cluster SIMS) depth profiles.

One embodiment provides a drug delivery composition wherein presence of active agent on at least a region of the surface of the composition is determined by time of flight secondary ion mass spectrometry (TOF-SIMS).

One embodiment provides a drug delivery composition wherein presence of active agent on at least a region of the surface of the composition is determined by atomic force microscopy (AFM).

One embodiment provides a drug delivery composition wherein presence of active agent on at least a region of the surface of the composition is determined by X-ray spectroscopy.

One embodiment provides a drug delivery composition of claim 1, wherein presence of active agent on at least a region of the surface of the composition is determined by electron microscopy.

One embodiment provides a drug delivery composition of claim 1, wherein presence of active agent on at least a region of the surface of the composition is determined by Raman spectroscopy.

Provided herein is a drug delivery composition comprising at least one bioabsorbable polymer and at least one active agent; wherein at least a portion of the active agent is in crystalline form; wherein the composition is in a form suitable for administration by injection and comprises polymer and active agent to provide a selected active agent controlled release profile.

One embodiment provides a composition wherein the biologically active agent is encapsulated in microparticles or nanoparticles.

One embodiment provides a composition wherein the bioabsorbable polymer has a glass transition temperature between 45 and 60° C.

One embodiment provides a composition wherein the bioabsorbable polymer gels at body temperature subsequent to heating the composition above the Tg temperature whereby the heated composition can be delivered in the form of bleb (flowable composition).

One embodiment provides a composition wherein the active agent is selected from rapamycin, a prodrug, a derivative, an analog, a hydrate, an ester, and a salt thereof.

One embodiment provides a composition wherein the active agent is selected from one or more of sirolimus, everolimus, zotarolimus and biolimus.

One embodiment provides a composition wherein the active agent comprises a macrolide immunosuppressive (limus) drug.

One embodiment provides a composition wherein the macrolide immunosuppressive drug comprises one or more of rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

One embodiment provides a composition wherein the active agent is at least 50% crystalline.

One embodiment provides a composition wherein the active agent is at least 75% crystalline.

One embodiment provides a composition wherein the active agent is at least 90% crystalline.

One embodiment provides a composition wherein the polymer comprises a PLGA copolymer.

One embodiment provides a composition wherein the composition comprises a first PLGA copolymer with a ratio of about 40:60 to about 60:40 and a second PLGA copolymer with a ratio of about 60:40 to about 90:10.

One embodiment provides a composition wherein the bioabsorbable polymer is selected from the group PLGA, PGA poly(glycolide), LPLA poly(1-lactide), DLPLA poly(dl-lactide), PCL poly(e-caprolactone) PDO, poly(dioxolane) PGA-TMC, 85/15 DLPLG p(dl-lactide-co-glycolide), 75/25 DLPL, 65/35 DLPLG, 50/50 DLPLG, TMC poly(trimethylcarbonate), p(CPP:SA) poly(1,3-bis-p-(carboxyphenoxy)propane-co-sebacic acid).

One embodiment provides a composition wherein between 25% and 45% of the total amount of active agent in the composition is released after 24 hours in vitro release in a 1:1 spectroscopic grade ethanol/phosphate buffer saline at pH 7.4 and 37° C.; wherein the amount of the active agent released is determined by measuring UV absorption at 278 nm by a diode array spectrometer.

One embodiment provides a composition wherein the composition has mucoadhesive properties.

One embodiment provides a composition wherein the mucoadhesive properties are provided by the bioabsorbable polymer.

One embodiment provides a composition wherein the composition is suitable for injection into a solid tumor to treat the neoplasm.

One embodiment provides a composition wherein the composition is suitable for injection or infusion into the bladder to treat bladder cancer.

One embodiment provides a composition wherein the composition is suitable for injection into vitreous humor of the eye to treat ocular disease.

One embodiment provides a composition wherein the composition is suitable for injection into the prostate gland to treat prostate cancer.

One embodiment provides a composition wherein the composition is suitable for injection into the nasal turbinates to treat chronic sinusitis.

One embodiment provides a composition wherein the composition is suitable for injection into intervention site.

One embodiment provides a composition wherein the intervention site is a wall of a body cavity.

One embodiment provides a composition wherein the body cavity is the result of a lumpectomy.

One embodiment provides a composition wherein the intervention site is a cannulized site within a subject.

One embodiment provides a composition wherein the intervention site is a sinus wall.

One embodiment provides a composition wherein the intervention site is located in the reproductive system of a subject.

One embodiment provides a composition wherein the composition is adapted to treat an ailment of the reproductive system.

One embodiment provides a composition wherein the intervention site is located in the urinary system of a subject.

One embodiment provides a composition wherein the composition is adapted to treat a disease of the urinary system.

One embodiment provides a composition wherein the intervention site is located at a tumor site.

One embodiment provides a composition wherein the tumor site is where a tumor is located.

One embodiment provides a composition wherein the tumor site is where a tumor was located prior to removal and/or shrinkage of the tumor.

One embodiment provides a composition wherein the intervention site is located in the ear.

One embodiment provides a composition wherein the intervention site is located in the esophagus.

One embodiment provides a composition wherein the intervention site is located in the larynx.

One embodiment provides a composition wherein the intervention site is a location of an injury.

One embodiment provides a composition wherein the intervention site is a location of an articulated joint.

One embodiment provides a composition wherein the intervention site is an infection site.

One embodiment provides a composition wherein the infection site is a site wherein an infection may occur, and wherein the active agent is capable of substantially preventing the infection.

One embodiment provides a composition wherein the infection site is a site wherein an infection has occurred, and wherein the active agent is capable of slowing spread of the infection.

One embodiment provides a composition wherein the intervention site is a surgery site.

One embodiment provides a composition wherein the intervention site is an ocular site.

One embodiment provides a composition wherein the composition is capable of at least one of: retarding healing, delaying healing, and preventing healing.

One embodiment provides a composition wherein the composition is capable of at least one of: retarding, delaying, and preventing the inflammatory phase of healing.

One embodiment provides a composition wherein the composition is capable of at least one of: retarding, delaying, and preventing the proliferative phase of healing.

One embodiment provides a composition wherein the active agent and the polymer are in the same layer, in separate layers, or form overlapping layers.

One embodiment provides a composition, wherein the plurality of layers comprise five layers deposited as follows: a first polymer layer, a first active agent layer, a second polymer layer, a second active agent layer and a third polymer layer.

One embodiment provides a composition wherein the plurality of layers comprise five layers deposited as follows: a first polymer layer, a first pharmaceutical agent layer, a second polymer layer, a second pharmaceutical agent layer and a third polymer layer.

One embodiment provides a composition wherein the composition releases the active agent at a selected therapeutic level over a period of at least 48 hours after injection.

One embodiment provides a composition wherein the composition releases the active agent with a linear release profile until about 30% of total content is released. One embodiment provides a composition wherein the composition releases the active agent with a linear release profile until about 50% of total content is released. One embodiment provides a composition wherein the composition releases the active agent with a linear release profile until about 70% of total content is released.

One embodiment provides a composition wherein the composition reduces or eliminates adverse toxic effect associated with oral formulation of the active agent.

One embodiment provides a composition wherein the composition is suitable for intramuscular injection or intraperitoneal injection.

One embodiment provides a composition wherein the composition is suitable for intraarticular, intrathecal, intravesicular or subcutaneous injection.

One embodiment provides a transdermal drug delivery system, comprising: (a) an impervious backing sheet; and (b) a reservoir containing a transdermal drug delivery composition, which comprises at least one bioabsorbable polymer and at least one active agent; wherein at least a portion of the active agent is in crystalline form; wherein the composition is in a form suitable for transdermal administration and comprises polymer and active agent layers formed to provide a selected active agent controlled release profile.

One embodiment provides a transdermal drug delivery system further comprising a microprotrusion member having a plurality of stratum corneum piercing microprotrusions thereon and being adapted for piercing the stratum corneum to improve transdermal flux of the composition.

One embodiment provides a transdermal drug delivery system wherein the reservoir comprises a jet dispenser comprising an orifice, and a container that holds and delivers the composition to said orifice for ejection therethrough.

One embodiment provides a transdermal drug delivery system further comprising a substantially planar substrate having an array of spaced apertures therein; and a plurality of microneedles projecting at angle from the plane in which the planar substrate lies.

One embodiment provides a transdermal drug delivery system wherein the at least one of the microneedles has a substantially rectangular cross-sectional shape in a plane parallel to the substrate.

One embodiment provides a transdermal drug delivery system wherein the at least one channel is open to two opposing surfaces of the microneedle.

One embodiment provides a transdermal drug delivery system wherein the at least one channel terminates in the body portion of the microneedle and does not extend into the tapered tip portion.

One embodiment provides a transdermal drug delivery system wherein the substrate and the microneedles comprise at least one biocompatible metal.

One embodiment provides a transdermal drug delivery system wherein the substrate and the microneedles comprise a stainless steel.

One embodiment provides an oral dosage form comprising a solid support core of a substantially water soluble, swellable or insoluble material and a composition comprising at least one bioabsorbable polymer and at least one active agent; wherein at least a portion of the active agent is in crystalline form; wherein the composition is in a form suitable for oral administration and comprises polymer and active agent layers formed to provide a selected active agent controlled release profile.

One embodiment provides an oral dosage further comprising a release-retarding composition layer.

One embodiment provides an oral dosage wherein the release retarding composition is a polymer having properties suitable for use in enteric coatings.

One embodiment provides an oral dosage further comprising one or more sub-coats beneath the release retarding composition layer.

One embodiment provides an oral dosage further comprising one or more over-coats above the release retarding composition layer.

One embodiment provides an oral dosage wherein the enteric coating further comprises a plasticizer.

One embodiment provides an oral dosage wherein the enteric polymer further comprises an anti-foaming agent.

One embodiment provides an oral dosage wherein the composition is coated with a top coat.

One embodiment provides an oral dosage wherein the top coat is hydroxypropylmethylcellulose.

One embodiment provides an oral dosage wherein the top coat is mucoadhesive.

One embodiment provides a drug delivery composition comprising at least one hydrogel and at least one active agent; wherein the active agent is present in crystalline form and is embedded in the hydrogel.

Another embodiment provides the drug delivery composition wherein the composition indicates the presence of said pharmaceutical agent in crystalline form upon analysis by an analytical method selected from: (a) X-ray spectroscopy, (b) scanning electron microscopy (SEM), (c) Raman spectrum, (d) Differential Scanning calorimetry (DSC), (e) Wide Angle X-ray Scattering (WAXS) spectroscopy, and (f) wide angle radiation scattering spectroscopy.

Another embodiment provides the drug delivery composition wherein curing of the hydrogel occurs in-vivo.

Another embodiment provides the drug delivery composition wherein the composition is formed to provide a selected active agent controlled release profile.

Another embodiment provides the drug delivery composition wherein the composition is in a form suitable for a mode of administration selected from: (a) injection, (b) intramuscular injection, (c) subcutaneous injection, (d) intrathecal injection, (e) intramuscular injection, (f) intraarticular injection, (g) intraperitoneal injection, (h) dermal administration, and (i) during a surgical procedure.

Another embodiment provides the drug delivery composition wherein the composition is adapted for delivery to at least one of: the front of the eye, the back of the eye, the location of a tumor, the location of cancerous cells, the location of cancerous tissue, the former location of cancerous tissue, the former location of cancerous cells, the former location of a tumor, the brain, a neurologic site, a location where inflammation is occurring, a location where inflammation may occur, a location where inflammation is expected to occur.

Another embodiment provides the drug delivery composition wherein the composition is adapted for dural repair.

Another embodiment provides the drug delivery composition wherein the composition is adapted to treat at least one of: intracranial aneurysms, tumors, and spinal disc disease.

Another embodiment provides the drug delivery composition wherein the composition is adapted to stop, slow, or prevent cervical spinal fluid leaks.

Another embodiment provides the drug delivery composition wherein the composition is capable of maintaining a seal under high pressures.

Another embodiment provides the drug delivery composition wherein the composition degrades as natural healing occurs.

Another embodiment provides the drug delivery composition wherein the composition acts as an adhesion barrier.

Another embodiment provides the drug delivery composition wherein the composition acts as a bandage.

Another embodiment provides the drug delivery composition wherein the composition is adapted to cover an opening in the eye and providing a protective barrier to the portion of the eye that is covered.

Another embodiment provides the drug delivery composition wherein the composition comprises voids.

Another embodiment provides the drug delivery composition wherein tissue ingrowth occurs in the voids as tissue heals.

Another embodiment provides the drug delivery composition wherein the composition is in the form of a mesh.

Another embodiment provides the drug delivery composition wherein the composition is adapted to seal tissue.

Another embodiment provides the drug delivery composition wherein the composition may be used in place of or in combination with tacks, staples, sutures, or O-rings in surgical procedures to seal tissue.

Another embodiment provides the drug delivery composition wherein the composition bioabsorbs or degrades as tissue grows into the voids of the mesh and into areas where the hydrogel has been degraded or absorbed.

Another embodiment provides the drug delivery composition wherein the hydrogel is biodegradable.

Another embodiment provides the drug delivery composition wherein the hydrogel is anti-microbial.

Another embodiment provides the drug delivery composition wherein the biologically active agent is encapsulated in microparticles or nanoparticles.

Another embodiment provides the drug delivery composition wherein the composition further comprises one or more agents that modulate the viscosity of the composition.

Another embodiment provides the drug delivery composition wherein the agent that modulates the viscosity of the composition lowers the viscosity of the composition at room temperature.

Another embodiment provides the drug delivery composition wherein the agent that modulates the viscosity of the composition increases the viscosity of the composition at temperatures above 35° C.

Another embodiment provides the drug delivery composition wherein the active agent is selected from rapamycin, a prodrug, a derivative, an analog, a hydrate, an ester, and a salt thereof.

Another embodiment provides the drug delivery composition wherein the active agent is selected from one or more of sirolimus, everolimus, zotarolimus and biolimus.

Another embodiment provides the drug delivery composition wherein the active agent comprises a macrolide immunosuppressive (limus) drug.

Another embodiment provides the drug delivery composition wherein the active agent is a macrolide immunosuppressive drug selected from one or more of rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′:E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

Another embodiment provides the drug delivery composition wherein the active agent is at least 50% crystalline, at least 75% crystalline, or at least 90% crystalline.

Another embodiment provides the drug delivery composition wherein the hydrogel comprises a polymer.

Another embodiment provides the drug delivery composition wherein the polymer comprises a PLGA copolymer.

Another embodiment provides the drug delivery composition wherein the polymer comprises a first PLGA copolymer with a ratio of about 40:60 to about 60:40 and a second PLGA copolymer with a ratio of about 60:40 to about 90:10.

Another embodiment provides the drug delivery composition wherein the polymer is selected from the group PLGA, PGA poly(glycolide), LPLA poly(1-lactide), DLPLA poly(dl-lactide), PCL poly(e-caprolactone) PDO, poly(dioxolane) PGA-TMC, 85/15 DLPLG p(dl-lactide-co-glycolide), 75/25 DLPL, 65/35 DLPLG, 50/50 DLPLG, TMC poly(trimethylcarbonate), p(CPP:SA) poly(1,3-bis-p-(carboxyphenoxy)propane-co-sebacic acid).

Another embodiment provides the drug delivery composition wherein between 25% and 45% of the total amount of active agent in the composition is released after 24 hours in vitro release in a 1:1 spectroscopic grade ethanol/phosphate buffer saline at pH 7.4 and 37° C.; wherein the amount of the active agent released is determined by measuring UV absorption at 278 nm by a diode array spectrometer.

Another embodiment provides the drug delivery composition wherein the composition is suitable for use in a mode of administration selected from: (a) injection into the bladder to treat bladder cancer, (b) injection into the prostate gland to treat prostate cancer, (c) injection into or near the vitreous humor of the eye to treat ocular disease, and (d) injection into the nasal turbinates to treat chronic sinusitis.

Another embodiment provides the drug delivery composition wherein the composition is suitable for injection into an intervention site wherein the intervention site is selected from: (a) the wall of a body cavity, (b) the wall of a body cavity resulting from partial or complete tumor removal, (c) a cannulized site within a subject, (d) a nasal turbinate, (e) within the reproductive system of a subject, (f) within the urinary system of a subject, (g) located at a tumor site, (f) a location in the ear, (g) a location in the esophagus, (h) a location in the larynx, (i) a location of an injury, (j) an infection site, (k) a surgery site, (l) an ocular site, (m) an inflammatory site, or (n) an arthritic joint.

Another embodiment provides the drug delivery composition wherein the composition is adapted to treat an ailment selected from: (a) an ailment of the reproductive system, (b) an ailment of the urinary system.

Another embodiment provides the drug delivery composition wherein the composition is adapted for delivery to at least one of an intervention site, an infection site, and an inflammatory site.

Another embodiment provides the drug delivery composition adapted for delivery to an intervention site wherein the intervention site is located in the urinary system of a subject.

Another embodiment provides the drug delivery composition adapted for delivery to an intervention site wherein the intervention site is located at a tumor site.

Another embodiment provides the drug delivery composition adapted for delivery to a tumor site wherein the tumor site is where a tumor is located.

Another embodiment provides the drug delivery composition adapted for delivery to a tumor site wherein the tumor site is where a tumor was located prior to removal and/or shrinkage of the tumor.

Another embodiment provides the drug delivery composition adapted for delivery to an intervention site wherein the intervention site is located in the ear.

Another embodiment provides the drug delivery composition adapted for delivery to an intervention site wherein the intervention site is located in the esophagus.

Another embodiment provides the drug delivery composition adapted for delivery to an intervention site wherein the intervention site is located in the larynx.

Another embodiment provides the drug delivery composition adapted for delivery to an intervention site wherein the intervention site is a location of an injury.

Another embodiment provides the drug delivery composition adapted for delivery to an intervention site wherein the intervention site is an infection site.

Another embodiment provides the drug delivery composition wherein the composition is adapted for delivery to an inflammatory site wherein the inflammatory site is a site wherein inflammation may occur, and wherein the active agent is capable of substantially preventing the inflammation.

Another embodiment provides the drug delivery composition wherein the composition is adapted for delivery to an inflammatory site wherein the inflammatory site is a site wherein inflammation may occur, and wherein the active agent is capable of substantially reducing the inflammation.

Another embodiment provides the drug delivery composition wherein the composition is adapted for delivery to an infection site wherein the infection site is a site wherein an infection may occur, and wherein the active agent is capable of substantially preventing the infection.

Another embodiment provides the drug delivery composition wherein the composition is adapted for delivery to an infection site wherein the infection site is a site wherein an infection has occurred, and wherein the active agent is capable of slowing spread of the infection.

Another embodiment provides the drug delivery composition wherein the composition is adapted for delivery to an intervention site wherein the intervention site is an ocular site.

Another embodiment provides the drug delivery composition wherein the composition is capable of at least one of: retarding healing, delaying healing, and preventing healing.

Another embodiment provides the drug delivery composition wherein the composition is capable of at least one of: retarding, delaying, and preventing the inflammatory phase of healing.

Another embodiment provides the drug delivery composition wherein the composition is capable of at least one of: retarding, delaying, and preventing the proliferative phase of healing.

Another embodiment provides the drug delivery composition wherein the active agent and the polymer are in the same layer, in separate layers, or form overlapping layers.

Another embodiment provides the drug delivery composition wherein the active agent is uniformly dispersed within the composition.

Another embodiment provides the drug delivery composition wherein the active agent release profile is a linear release profile until 30% of the total content of active agent is released.

Another embodiment provides the drug delivery composition wherein the active agent release profile is a linear release profile until 50% of the total content of active agent is released.

Another embodiment provides the drug delivery composition wherein the composition releases the active agent at a selected therapeutic level over a period of at least 48 hours after injection.

Another embodiment provides the drug delivery composition wherein the composition reduces or eliminates adverse toxic effect associated with oral formulation of the active agent.

Another embodiment provides the drug delivery composition wherein the composition is suitable for intramuscular injection or intraperitoneal injection.

Another embodiment provides the drug delivery composition wherein the active agent is delivered transdermally from the composition at a selected active agent release profile.

Another embodiment provides the transdermal drug delivery composition further comprising a microprotrusion member having a plurality of stratum corneum piercing microprotrusions thereon and being adapted for piercing the stratum corneum to improve transdermal flux of the composition.

Another embodiment provides the transdermal drug delivery composition wherein the composition releases the active agent at a selected therapeutic level over a period of at least 48 hours after application of the composition.

Another embodiment provides the transdermal drug delivery composition wherein the composition reduces or eliminates adverse toxic effect associated with oral formulation of the active agent.

Another embodiment provides the drug delivery composition wherein the composition is adapted for delivery intra-articularly.

Another embodiment provides the drug delivery composition wherein the hydrogel comprises a hydrogel composition that is derived from an activated polyalkylene glycol diacid derivative and a crosslinking agent.

Another embodiment provides the drug delivery composition comprising a hydrogel composition wherein the activated polyalkylene diacid derivative is represented by formula (I):

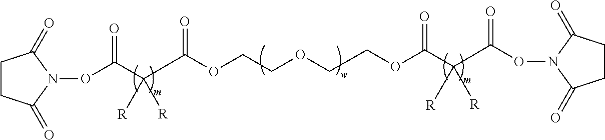

wherein, independently for each occurrence, R is H or lower alkyl; m is 2-20 inclusive; and w is 5 to 1,000 inclusive.

Another embodiment provides the drug delivery composition comprising a hydrogel composition wherein the crosslinking agent is a polyalkyleneimine or trilysine.

Another embodiment provides the drug delivery composition comprising a hydrogel composition wherein the crosslinking agent is polyethyleneimine having a molecular weight of about 2000.

Another embodiment provides the drug delivery composition comprising a hydrogel composition wherein the crosslinking agent is trilysine.

Another embodiment provides the drug delivery composition comprising a hydrogel component of formula (I) wherein m is 2-10 inclusive.

Another embodiment provides the drug delivery composition comprising a hydrogel component of formula (I) wherein m is 2.

Another embodiment provides the drug delivery composition comprising a hydrogel component of formula (I) wherein m is 3.

Another embodiment provides the drug delivery composition comprising a hydrogel component of formula (I) wherein m is 4.

Another embodiment provides the drug delivery composition comprising a hydrogel component of formula (I) wherein m is 6.

Another embodiment provides the drug delivery composition comprising a hydrogel component of formula (I) wherein m is 8.

Another embodiment provides the drug delivery composition comprising a hydrogel component of formula (I) wherein w is 20 to 120 inclusive.

Another embodiment provides the drug delivery composition comprising a hydrogel component of formula (I) wherein w is 120 to 250 inclusive.

Another embodiment provides the drug delivery composition comprising a hydrogel composition wherein the activated polyalkylene glycol diacid derivative is R-PEGₙ-R; wherein n represents the number average molecular weight of the PEG and is about 2000 to about 12,000 inclusive; and R is SS, SG, SA, SSub, or SSeb.

Another embodiment provides the drug delivery composition comprising a hydrogel composition wherein the activated polyalkylene glycol diacid derivative is PEG₃₃₅₀-(SS)₂.

Another embodiment provides the drug delivery composition comprising a hydrogel composition wherein the activated polyalkylene glycol diacid derivative is (SS)-PEG₃₃₅₀-(SS).

Another embodiment provides the drug delivery composition comprising a hydrogel composition wherein the activated polyalkylene glycol diacid derivative is PEG₃₃₅₀-(SG)₂.

Another embodiment provides the drug delivery composition comprising a hydrogel composition wherein the activated polyalkylene glycol diacid derivative is (SG)-PEG₃₃₅₀-(SG).

Another embodiment provides the drug delivery composition comprising a hydrogel composition wherein the activated polyalkylene glycol diacid derivative is PEG₃₃₅₀-(SA)₂.

Another embodiment provides the drug delivery composition comprising a hydrogel composition wherein the activated polyalkylene glycol diacid derivative is (SA)-PEG₃₃₅₀-(SA).

Another embodiment provides the drug delivery composition comprising a hydrogel composition wherein the activated polyalkylene glycol diacid derivative is PEG₃₃₅₀-(SSeb)₂.

Another embodiment provides the drug delivery composition comprising a hydrogel composition wherein the activated polyalkylene glycol diacid derivative is (SSeb)-PEG₃₃₅₀-(SSeb).

Another embodiment provides the drug delivery composition comprising a hydrogel composition wherein the activated polyalkylene glycol diacid derivative is PEG₃₃₅₀-(SSub)₂.

Another embodiment provides the drug delivery composition comprising a hydrogel composition wherein the activated polyalkylene glycol diacid derivative is (SSub)-PEG₃₃₅₀-(SSub).

Another embodiment provides the drug delivery composition comprising a hydrogel component wherein the weight percent crosslinker is between about 5% and about 50%.

Another embodiment provides the drug delivery composition comprising a hydrogel component wherein the weight percent crosslinker is about 15%.

Another embodiment provides the drug delivery composition comprising a hydrogel component wherein the ratio of activated esters on the polyalkylene glycol diacid derivatives to primary amines on the crosslinking agent is in the range from about 0.10:1 to about 10:1.

Another embodiment provides the drug delivery composition comprising a hydrogel component wherein the ratio of activated esters on the polyalkylene glycol diacid derivatives to primary amines on the crosslinking agent is in the range from about 0.75:1 to about 1.3:1.

Another embodiment provides the drug delivery composition comprising a hydrogel component wherein R is methyl or H.

Another embodiment provides the drug delivery composition comprising a hydrogel component wherein R is H.

### Injectable Dosage Forms

This example will use a rodent (rat) model to demonstrate that the injectable drug deposition technology results in more constant rapamycin blood levels than seen with once-daily oral dosing. A rat model of cardiac allograft transplantation has been used previously to assess the effectiveness of continuous intravenous infusion of rapamycin compared to oral dosing. This previous study provides data on how blood levels of rapamycin correlate with anti-rejection effectiveness and establishes an effective dose range for the rat based on that data.

### Objective:

This study will compare the injectable drug deposition technology to once-daily oral dosing of rapamycin with regard to maintaining a more constant level of rapamycin in the blood. Three different doses of injectable rapamycin will be used to begin to develop an understanding of the pharmacokinetics of this mode of administration. The oral dose used will be 4.9 mg/kg/day. This has been shown to result in prolonged allograft survival in 70% of rats treated. As this study is only assessing the blood levels of drug, transplantation of allograft tissue is not needed.

### Method:

Adult male Wistar Furth rats, 7-9 weeks old will be used to better correlate with results from previous studies evaluating rapamycin dosing and effectiveness. An injectable formulation of rapamycin will be prepared as described in the previous above under Sample Preparation. The formulation will be modified to allow for three different doses of drug to be delivered from a depot created with approximately a 1 ml volume of injectate. Each formulation will be loaded into an individual syringe and injected into the subcutaneous space of a rat (n=3 for each formulation). Three rats will receive once daily dosing of rapamycin (4.9 mg/kg) by oral gavage.

The duration of the study will be 14 days with 0.3 ml blood draws from either a tail vein or indwelling catheter every other day immediately before and 1.5 hours after daily dosing in the animals receiving oral drug and once each day from animals that received a injectable dose of drug at the beginning of the study. The amount of blood drawn may change based on results of preliminary studies to ascertain the minimum sample volume needed to accurately assess drug content. Blood concentrations of rapamycin will be determined based on established methods.

The data collected will demonstrate the variability in blood drug levels between peak (1.5 hours post oral dosing) and trough (24 hours after once daily dosing) values and also from day to day. The hypothesis to be tested is whether an injectable depot of drug using the formulations disclosed herein will result in less variability in systemic drug levels over time. It is anticipated that the drug-polymer formulation will allow for a relatively constant rate of drug delivery. This data will also allow for a determination of the amount and duration of drug delivery from a single injectable depot containing 1 ml of a specific formulation. Variability of drug levels will be assessed using appropriate statistical analyses.

### Lipophilic and Biologic Compositions

Provided herein is a drug delivery composition comprising a lipophilic agent and a polymer wherein the lipophilic agent exhibits sustained release and wherein there is less than 10% agent release within the first hour of elution.

Provided herein is a drug delivery composition comprising a lipophilic agent and a polymer wherein the elution profile is substantially linear, and wherein there is less than 10% agent release within the first hour of elution.

In some embodiments, the elution profile is determined through in-vitro testing. In some embodiments, the elution profile is determined through in-vivo testing. In some embodiments, sustained release comprises the lipophilic agent releasing over a period of >3 hours, >24 hours, >1 week, >3 months, >6 months, and/or >12 months. In some embodiments, there is less than 5% agent release within the first hour of elution. In some embodiments, no agent is delivered in the first hour of elution. In some embodiments, the in-vitro elution profile exhibits no burst of elution in the first hour of elution, in the first 3 hours of elution, in the first 6 hours of elution, in the first 9 hours of elution, in the first 12 hours of elution, in the first 18 hours of elution, and/or in the first day of elution. In some embodiments, the elution profile is substantially linear once a detectable amount of drug is eluted.

Provided herein is a drug delivery composition comprising a lipophilic agent and a polymer wherein the PK profile varies only within 10% of an average PK profile for the composition.

Provided herein is a drug delivery composition comprising a lipophilic agent and a polymer wherein the AUC (area under the curve which is the integral of the concentration-time curve after a single dose or in steady state) of the PK profile of the composition varies only within 10% of an average AUC for the composition.

Provided herein is a drug delivery composition comprising a lipophilic agent and a polymer wherein the Cmax (which is the peak plasma concentration of a drug after administration) of the PK profile of the composition varies only within 10% of an average Cmax for the composition.

Provided herein is a drug delivery composition comprising a lipophilic agent and a polymer wherein the Tmax (which is the time it takes to reach Cmax) of the PK profile varies only within 10% of an average Tmax for the composition.

In some embodiments, the PK profile of the composition is linear such that the linear regression fit to the in-vivo PK data has a R̂2 value of >0.8, or such that the linear regression fit to the in-vitro PK data has a R̂2 value of >0.8.

In some embodiments, the lipophilic agent comprises a limus drug, mTOR inhibitors, antibiotic agents, and/or immunosuppressive agents.

In some embodiments, the polymer comprises any one or more of a linear polymer, branched polymer, dendritic polymer, liquid crystalline polymer, amorphous polymer, semi-crystalline polymer, block co-polymer, tri-block copolymer, graft copolymer, polymer blend, ionomeric polymers, absorbable polymer, and bio-polymer.

In some embodiments, the polymer comprises a tri-block polymer with two hydrophilic chains connected by a hydrophobic chain. In some embodiments, the polymer comprises a tri-block polymer with two hydrophobic chains connected by a hydrophilic chain.

In some embodiments, the polymer comprises a pluronic polymer. In some embodiments, the pluronic polymer comprises a biocompatible reverse thermosensitive polymer.

In some embodiments, the polymer comprises a poloxamer or a poloxamine, or a combination thereof. In some embodiments, the polymer comprises poloxamer 407, poloxamer 338, poloxamer 118, poloxamine 1107 or poloxamine 1307, or a combination thereof. In some embodiments, the polymer comprises block copolymers, random copolymers, graft polymers, branched copolymers, or a combination thereof. In some embodiments, the polymer comprises polyoxyalkylene block copolymer. In some embodiments, the polymer comprises at least one purified inverse thermosensitive polymer selected from the group consisting of purified poloxamers and purified poloxamines. In some embodiments, the composition has a transition temperature of between about 10° C. and about 40° C. In some embodiments, the composition has a transition temperature of between about 15° C. and about 30° C. In some embodiments, the volume of said composition at physiological temperature is about 80% to about 120% of its volume below its transition temperature. In some embodiments, the volume of said composition at physiological temperature is about 80% to about 120% of its volume below its transition temperature; and said composition has a transition temperature of between about 10° C. and about 40° C. In some embodiments, the volume of said composition at physiological temperature is about 80% to about 120% of its volume below its transition temperature; and said composition has a transition temperature of between about 15° C. and about 30° C. In some embodiments, the volume of said composition at physiological temperature is about 80% to about 120% of its volume below its transition temperature; said composition has a transition temperature of between about 10° C. and about 40° C.; and said composition comprises at least one purified inverse thermosensitive polymer selected from the group consisting of poloxamers and poloxamines. In some embodiments, the volume of said composition at physiological temperature is about 80% to about 120% of its volume below its transition temperature; said composition has a transition temperature of between about 15° C. and about 30° C.; and said composition comprises at least one purified inverse thermosensitive polymer selected from the group consisting of poloxamers and poloxamines. In some embodiments, the composition comprises about 50% to about 35% of said inverse thermosensitive polymer. In some embodiments, the composition comprises about 5% to about 30% of said inverse thermosensitive polymer. In some embodiments, the inverse thermosensitive polymer has a polydispersity index from about 1.5 to about 1.0. In some embodiments, the inverse thermosensitive polymer has a polydispersity index from about 1.2 to about 1.0.

In some embodiments, the composition is a gel at a first temperature and a liquid at a second temperature, wherein the second temperature is lower than the first temperature. In some embodiments, the composition is a gel at body temperature. In some embodiments, the first temperature is at most 5° C. higher, 3° C. higher, at most 2° C. higher, at most 1° C. higher, at most 0.5° C. higher, at most 0.3° C. higher, or at most 0.2° C. higher than the second temperature.

In some embodiments, the composition at physiological conditions after 24 hours contains >5% aqueous mass. In some embodiments, the composition has a sol (water) fraction of a hydrogel at physiological conditions after 24 hours. In some embodiments, the sol fraction is the fractional increase in the weight of the composition due to water absorption. In some embodiments, the composition is a hydrogel and/or has hydrogel properties.

In some embodiments, at least a portion of the lipophilic agent is crystalline or semi-crystalline. In some embodiments, at least 50%, 60%, 70%, 75%, 80%, 85%, 90%, 92%, 95%, 98%, 99%, 99.0%, 99.5%, or 99% of the agent in the composition is in crystalline or semi-crystalline form.

In some embodiments, the composition is formulated for use in, treatment of, or diagnosis of: chronic diseases; cancer; neurologic ailments, conditions, disorders, or diseases; pain, reproductive ailments, conditions, disorders, or diseases, or as part of a reproductive therapies or treatment; opthamalogic ailments, conditions, disorders, or diseases; GI ailments, conditions, disorders, or diseases; orthopedic ailments, conditions, disorders, or diseases; surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications.

In some embodiments, the composition is incorporated into a system comprising a substrate that carries the composition to the administration site or delivery site or treatment site. In some embodiments, the substrate comprises a medical implant, diagnostic device, interventional device, and/or surgical tool. In some embodiments, the substrate is a device, tool, or implant for use in orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications. In some embodiments, the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology.

Provided herein is a drug delivery composition comprising a biological agent and a polymer wherein the biological agent exhibits sustained release and wherein there is less than 10% agent release within the first hour of elution.

Provided herein is a drug delivery composition comprising a biological agent and a polymer wherein the elution profile is substantially linear, and wherein there is less than 10% agent release within the first hour of elution.

In some embodiments, the elution profile is determined through in-vitro testing. In some embodiments, the elution profile is determined through in-vivo testing. In some embodiments, sustained release comprises the biological agent releasing over a period of >3 hours, >24 hours, >1 week, >3 months, >6 months, and/or >12 months. In some embodiments, there is less than 5% agent release within the first hour of elution. In some embodiments, no agent is delivered in the first hour of elution. In some embodiments, the in-vitro elution profile exhibits no burst of elution in the first hour of elution, in the first 3 hours of elution, in the first 6 hours of elution, in the first 9 hours of elution, in the first 12 hours of elution, in the first 18 hours of elution, and/or in the first day of elution. In some embodiments, the elution profile is substantially linear once a detectable amount of agent is eluted.

Provided herein is a drug delivery composition comprising a biological agent and a polymer wherein the PK profile varies only within 10% of an average PK profile for the composition.

Provided herein is a drug delivery composition comprising a biological agent and a polymer wherein the AUC (area under the curve which is the integral of the concentration-time curve after a single dose or in steady state) of the PK profile of the composition varies only within 10% of an average AUC for the composition.

Provided herein is a drug delivery composition comprising a biological agent and a polymer wherein the Cmax (which is the peak plasma concentration of a drug after administration) of the PK profile of the composition varies only within 10% of an average Cmax for the composition.

Provided herein is a drug delivery composition comprising a biological agent and a polymer wherein the Tmax (which is the time it takes to reach Cmax) of the PK profile varies only within 10% of an average Tmax for the composition.

In some embodiments, the PK profile of the composition is linear such that the linear regression fit to the in-vivo PK data has a R̂2 value of >0.8, or such that the linear regression fit to the in-vitro PK data has a R̂2 value of >0.8.

In some embodiments, the biological agent comprises peptides, proteins, enzymes, derivatives and analogs of natural peptides, proteins and enzymes, glycoproteins, nucleic acids (including deoxyribonucleotide or ribonucleotide polymers in either single or double stranded form, and unless otherwise limited, encompasses known analogues of natural nucleotides that hybridize to nucleic acids in a manner similar to naturally occurring nucleotides), antisense nucleic acids, fatty acids, antimicrobials, vitamins, hormones, steroids, lipids, polysaccharides, carbohydrates, gene therapies, RNA, siRNA, and/or cellular therapies (for non-limiting example, stem cells or T-cells), or combinations thereof.

In some embodiments, the composition is a gel at a first temperature and a liquid at a second temperature, wherein the second temperature is lower than the first temperature. In some embodiments, the first temperature is at most 5° C. higher, 3° C. higher, at most 2° C. higher, at most 1° C. higher, at most 0.5° C. higher, at most 0.3° C. higher, or at most 0.2° C. higher than the second temperature.

In some embodiments, the composition is a gel at body temperature.

In some embodiments, the polymer comprises any one or more of a linear polymer, branched polymer, dendritic polymer, liquid crystalline polymer, amorphous polymer, semi-crystalline polymer, block co-polymer, tri-block copolymer, graft copolymer, polymer blend, ionomeric polymers, absorbable polymer, and bio-polymer.

In some embodiments, the polymer comprises a tri-block polymer with two hydrophilic chains connected by a hydrophobic chain. In some embodiments, the polymer comprises a tri-block polymer with two hydrophobic chains connected by a hydrophilic chain.

In some embodiments, the polymer comprises a pluronic polymer. In some embodiments, the pluronic polymer comprises a biocompatible reverse thermosensitive polymer.

In some embodiments, the polymer comprises a poloxamer or a poloxamine, or a combination thereof. In some embodiments, the polymer comprises poloxamer 407, poloxamer 338, poloxamer 118, poloxamine 1107 or poloxamine 1307, or a combination thereof. In some embodiments, the polymer comprises block copolymers, random copolymers, graft polymers, branched copolymers, or a combination thereof. In some embodiments, the polymer comprises polyoxyalkylene block copolymer. In some embodiments, the polymer comprises at least one purified inverse thermosensitive polymer selected from the group consisting of purified poloxamers and purified poloxamines. In some embodiments, the composition has a transition temperature of between about 10° C. and about 40° C. In some embodiments, the composition has a transition temperature of between about 15° C. and about 30° C. In some embodiments, the volume of said composition at physiological temperature is about 80% to about 120% of its volume below its transition temperature. In some embodiments, the volume of said composition at physiological temperature is about 80% to about 120% of its volume below its transition temperature; and said composition has a transition temperature of between about 10° C. and about 40° C. In some embodiments, the volume of said composition at physiological temperature is about 80% to about 120% of its volume below its transition temperature; and said composition has a transition temperature of between about 15° C. and about 30° C. In some embodiments, the volume of said composition at physiological temperature is about 80% to about 120% of its volume below its transition temperature; said composition has a transition temperature of between about 10° C. and about 40° C.; and said composition comprises at least one purified inverse thermosensitive polymer selected from the group consisting of poloxamers and poloxamines. In some embodiments, the volume of said composition at physiological temperature is about 80% to about 120% of its volume below its transition temperature; said composition has a transition temperature of between about 15° C. and about 30° C.; and said composition comprises at least one purified inverse thermosensitive polymer selected from the group consisting of poloxamers and poloxamines. In some embodiments, the composition comprises about 50% to about 35% of said inverse thermosensitive polymer. In some embodiments, the composition comprises about 5% to about 30% of said inverse thermosensitive polymer. In some embodiments, the inverse thermosensitive polymer has a polydispersity index from about 1.5 to about 1.0. In some embodiments, the inverse thermosensitive polymer has a polydispersity index from about 1.2 to about 1.0.

In some embodiments, the composition at physiological conditions after 24 hours contains >5% aqueous mass. In some embodiments, the composition has a sol (water) fraction of a hydrogel at physiological conditions after 24 hours. In some embodiments, the sol fraction is the fractional increase in the weight of the composition due to water absorption.

In some embodiments, the composition is a hydrogel and/or has hydrogel properties.

In some embodiments, the biological agent is in an active form, wherein active form comprises having a degree of secondary, tertiary and/or quaternary structure upon which the activity of the agent depends. In some embodiments, at least 50%, 60%, 70%, 75%, 80%, 85%, 90%, 92%, 95%, 98%, 99%, 99.0%, 99.5%, or 99% of the agent in the composition is in active form.

In some embodiments, the composition is formulated for use in, treatment of, or diagnosis of: chronic diseases; cancer; neurologic ailments, conditions, disorders, or diseases; pain, reproductive ailments, conditions, disorders, or diseases, or as part of a reproductive therapies or treatment; opthamalogic ailments, conditions, disorders, or diseases; GI ailments, conditions, disorders, or diseases; orthopedic ailments, conditions, disorders, or diseases; surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications.

In some embodiments, the composition is incorporated into a system comprising a substrate that carries the composition to the administration site or delivery site or treatment site. In some embodiments, the substrate comprises a medical implant, diagnostic device, interventional device, and/or surgical tool. In some embodiments, the substrate is a device, tool, or implant for use in orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications.

In some embodiments, the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology.

As used herein, the term “about,” unless otherwise defined for the aspect to which it refers, means variations of any of 0.01%, 0.05%, 0.1%, 0.5%, 1%, 2%, 5%, 10%, 15%, 20%, 25%, 30%, and 50% on either side of the aspect target or on a single side of the aspect target, depending on the embodiment. When referring to an aspect that is expressed as a percent, the term about does not generally refer to a percent of the percent, but rather a range about the percent—unless otherwise stated. For non-limiting example, if an aspect was “about 5.0%” and the variation for about was 0.5% (depending on the embodiment), this could mean 5.0% plus or minus 0.5%—equating to a range of 4.5% to 5.5%.

### EXAMPLES

The following examples are provided to illustrate selected embodiments. They should not be considered as limiting the scope of the invention, but merely as being illustrative and representative thereof. For each example listed herein, multiple analytical techniques may be provided. Any single technique of the multiple techniques listed may be sufficient to show the parameter and/or characteristic being tested, or any combination of techniques may be used to show such parameter and/or characteristic. Those skilled in the art will be familiar with a wide range of analytical techniques for the characterization of drug/polymer compositions. Techniques presented here, but not limited to, may be used to additionally and/or alternatively characterize specific properties of the compositions with variations and adjustments employed which would be obvious to those skilled in the art.

### Example 1

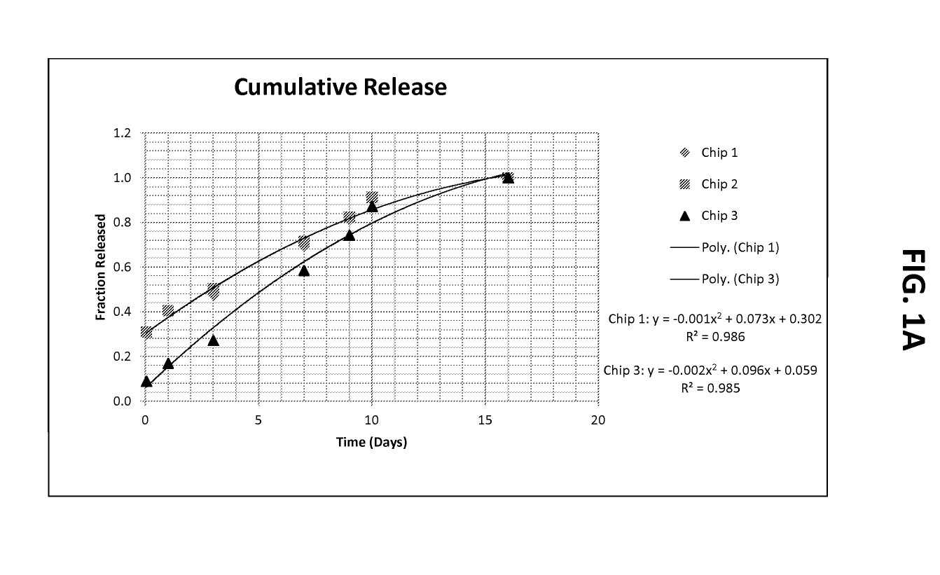

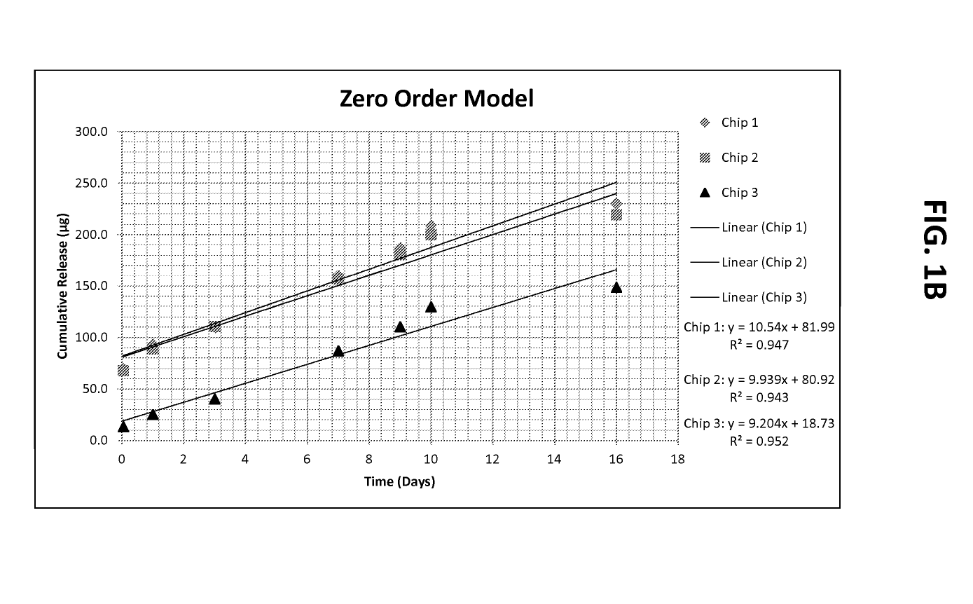

A mixture of 0.01 grams of rapamycin to 1 gram of PEG were mixed vigorously with a vortex mechanical mixer. A solvent in which the PEG is soluble and the rapamycin is insoluble was then added. The mixture was mixed again and then sprayed from a dual barrel syringe onto a 316 SS casting form. The second syringe was filled with a polymerizing solvent. Three hydrogel chips were cut from the same sheet with a scalpel. Chips 1 and 2 were approximately the size while chip 3 was smaller. The in vitro pharmaceutical agent elution profile was determined by contacting each chip with an elution media comprising ethanol (5%) wherein the pH of the media is about 7.4 and wherein the chip was contacted with the elution media at a temperature of about 37° C. The elution media was removed from chip contact at least at 1d, 3d, 7d, 9d, 10d, and up to 16 days (See FIGS. 1A, 1B, 2, and 3). The elution media was then assayed using a UV-Vis for determination of the pharmaceutical agent content (in absolute amount and cumulative amount eluted). The elution media was replaced at each time point with fresh elution media to avoid saturation of the elution media. Calibration standards containing known amounts of drug were also held in elution media for the same durations as the samples and assayed by UV-Vis at each time point to determine the amount of drug eluted at that time (in absolute amount and as a cumulative amount eluted), compared to a blank comprising spectroscopic grade ethanol (95%). Elution profiles are shown in FIGS. 1A, 1B, 2, and 3. Table 1 shows for each chip (chip 1, chip 2, chip 3), the absolute amount of rapamycin eluted in micrograms. Table 2 shows for each chip (chip 1, chip 2, chip 3), the cumulative fraction of rapamycin eluted.

TABLE 1
Mass Release

Mass Release (μg)
Chip

| Days | 1 | 2 | 3 | Control |
|---|---|---|---|---|
| 0.041667 | 69.7 | 67.9 | 13.3 | 0 |
| 1 | 23.7 | 21.0 | 11.9 | 0 |
| 3 | 16.4 | 21.7 | 15.2 | 0 |
| 7 | 50.6 | 46.2 | 46.6 | 0 |
| 9 | 26.9 | 23.9 | 23.5 | 0 |
| 10 | 21.7 | 19.4 | 19.2 | 0 |
| 16 | 21.2 | 19.2 | 19.1 | 0 |

TABLE 2
Cumulative Fraction Release
Cumulative Release

| | | |
|---|---|---|
| Chip 1 | Chip 2 | Chip 3 |

| Mass | | Mass | | Mass | |
|---|---|---|---|---|---|
| (mg) | Percentage | (mg) | Percentage | (mg) | Percentage |
| 69.7 | 0.3 | 67.9 | 0.3 | 13.3 | 0.1 |
| 93.4 | 0.4 | 88.9 | 0.4 | 25.2 | 0.2 |
| 109.7 | 0.5 | 110.6 | 0.5 | 40.4 | 0.3 |
| 160.4 | 0.7 | 156.8 | 0.7 | 87.0 | 0.6 |
| 187.3 | 0.8 | 180.7 | 0.8 | 110.5 | 0.7 |
| 209.0 | 0.9 | 200.1 | 0.9 | 129.7 | 0.9 |
| 230.2 | 1.0 | 219.3 | 1.0 | 148.8 | 1.0 |

FIG. 1A depicts the cumulative fraction release of rapamycin from hydrogel chips from the data in Table 2, where the elution profile was determined by static elution media of 5% EtOH/water, pH 7.4, 37° C. via a UV-Vis test method as described in Example 1 of hydrogel chips described therein. FIG. 1A shows that hydrogel chips 1 and 2 had nearly identical elution profiles. At the first time point (t=1 hour) about 30% of the total rapamycin eluted from chips 1 and 2. Following this initial elution of rapamycin from the hydrogel chips 1 and 2, the rapamycin exhibited sustained release over the course of 16 days. After the initial release, the rapamycin elution followed the polynomial y=−000.1x²+0.073x+0.302. For hydrogel chip 3, about 10% of the rapamycin eluted at one hour. Following this initial elution of rapamycin from the hydrogel chip 3, the rapamycin exhibited sustained release over the course of 16 days. After the initial release, the rapamycin elution from chip 3 followed the polynomial y=−000.2x²+0.096x+0.059.

FIG. 1B depicts the cumulative mass release of rapamycin from hydrogel chips from the data in Table 1, where the elution profile was determined by static elution media of 5% EtOH/water, pH 7.4, 37° C. via a UV-Vis test method as described in Example 1 of hydrogel chips described therein. FIG. 1B shows that hydrogel chips 1 and 2 had nearly identical elution profiles. At the first time point (t=1 hour), 69.7 μg and 67.9 μg of rapamycin eluted from hydrogel chips 1 and 2, respectively. For hydrogel chip 3, 67.9 μg of rapamycin eluted from the chip at one hour. The elution profile for all three hydrogel chips was substantially linear (chip 1, R²=0.947; chip 2, R²=0.943; chip 3, R²=0.952).

TABLE 3
Korsmeyer-Pappas Model

Korsmeyer-Pappas
Model

| | Chip 1 | Chip 2 | Chip 3 |
|---|---|---|---|
| | log(Cum % | log(Cum % | log(Cum % |
| log(time) | Release) | Release) | Release) |
| −1.38 | −0.52 | −0.51 | −1.05 |
| 0.00 | −0.39 | −0.39 | −0.77 |
| 0.48 | −0.32 | −0.30 | −0.57 |
| 0.85 | −0.16 | −0.15 | −0.23 |
| 0.95 | −0.09 | −0.08 | −0.13 |
| 1.00 | −0.04 | −0.04 | −0.06 |
| 1.20 | 0.00 | 0.00 | 0.00 |

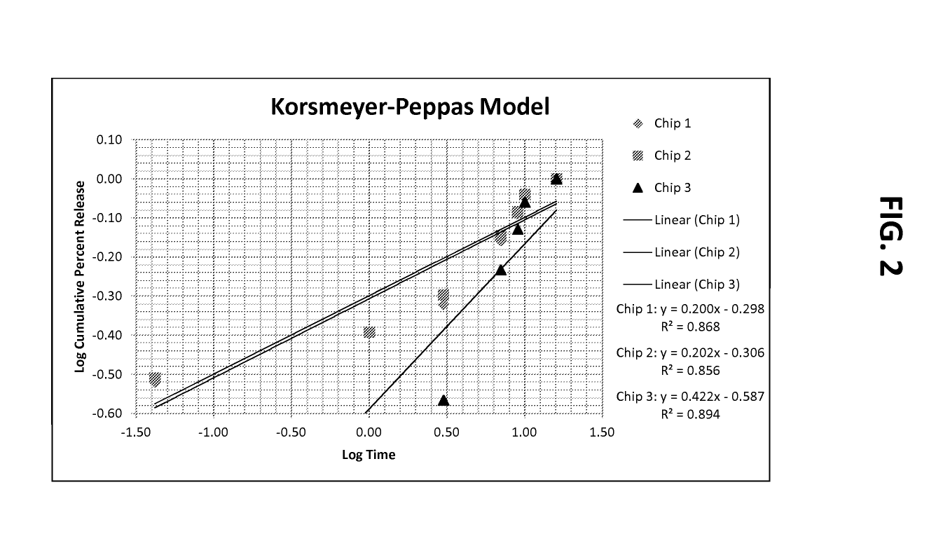

FIG. 2 depicts the log cumulative percent release (Korsmeyer-Pappas model of drug release) of rapamycin from hydrogel chips from the data in Table 3, where the elution profile was determined by static elution media of 5% EtOH/water, pH 7.4, 37° C. via a UV-Vis test method as described in Example 1 of hydrogel chips described therein. FIG. 2 shows that hydrogel chips 1 and 2 had nearly identical drug release profiles.

TABLE 4
Higuchi Model

| | | | |
|---|---|---|---|
| Higuchi Model | Chip 1 | Chip 2 | Chip 3 |

| | Sq Root | Cum % | Cum % | Cum % |
|---|---|---|---|---|
| Days | Time | Release | Release | Release |
| 0.04 | 0.20 | 0.30 | 0.31 | 0.09 |
| 1.00 | 1.00 | 0.41 | 0.41 | 0.17 |
| 3.00 | 1.73 | 0.48 | 0.50 | 0.27 |
| 7.00 | 2.65 | 0.70 | 0.72 | 0.58 |
| 9.00 | 3.00 | 0.81 | 0.82 | 0.74 |
| 10.00 | 3.16 | 0.91 | 0.91 | 0.87 |
| 16.00 | 4.00 | 1.00 | 1.00 | 1.00 |

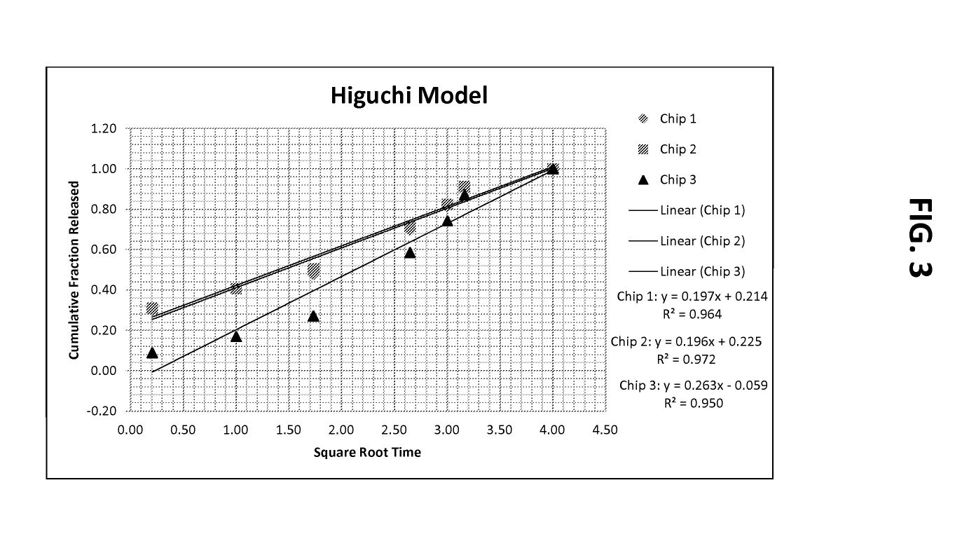

FIG. 3 depicts the cumulative fraction release (Higuchi model of drug release; Higuchi, J. Pharm. Sci., 1964, vol 84, 1461) of rapamycin from hydrogel chips from the data in Table 4, where the elution profile was determined by static elution media of 5% EtOH/water, pH 7.4, 37° C. via a UV-Vis test method as described in Example 1 of hydrogel chips described therein. FIG. 3 shows that hydrogel chips 1 and 2 had nearly identical elution profiles. The elution profile for all three hydrogel chips was substantially linear (chip 1, R²=0.964; chip 2, R²=0.972; chip 3, R²=0.950).

### Sample Preparation

Generally speaking, compositions prepared for in-vivo models are prepared as herein. Nevertheless, modifications for a given analytical method are presented within the examples shown, and/or would be obvious to one having skill in the art. Thus, numerous variations, changes, and substitutions will now occur to those skilled in the art without departing from the invention. It should be understood that various alternatives to the embodiments of the invention described herein and examples provided may be employed in practicing the invention and showing the parameters and/or characteristics described.

### Compositions for Controlled Release Dosage Forms

In some examples, the composition process is PDPDP (Polymer, sinter, Drug, Polymer, sinter, Drug, Polymer, sinter) using deposition of drug in dry powder form and deposition of polymer particles by RESS methods and equipment described herein. In the illustrations herein, resulting in a 3-layer composition comprising polymer (for example, PLGA) in the first layer, drug (for example, rapamycin) in a second layer and polymer in the third layer, where a portion of the third layer is substantially drug free (e.g. a sub-layer within the third layer having a thickness equal to a fraction of the thickness of the third layer). As described layer, the middle layer (or drug layer) may be overlapping with one or both first (polymer) and third (polymer) layer. The overlap between the drug layer and the polymer layers is defined by extension of polymer material into physical space largely occupied by the drug. The overlap between the drug and polymer layers may relate to partial packing of the drug particles during the formation of the drug layer. When crystal drug particles are deposited on top of the first polymer layer, voids and or gaps may remain between dry crystal particles. The voids and gaps are available to be occupied by particles deposited during the formation of the third (polymer) layer. Some of the particles from the third (polymer) layer may rest in the vicinity of drug particles in the second (drug) layer. When the sintering step is completed for the third (polymer) layer, the third polymer layer particles fuse to form a continuous film that forms the third (polymer) layer. In some embodiments, the third (polymer) layer however will have a portion along the longitudinal axis of the stent whereby the portion is free of contacts between polymer material and drug particles. The portion of the third layer that is substantially of contact with drug particles can be as thin as 1 nanometer.

The composition may be analyzed (for example, for analysis of crystallinity of the active agent). The composition may be sliced into sections which may be visualized using the surface composition techniques presented herein or other techniques known in the art for surface composition analysis (or other characteristics, active agent distribution, for example).

The compositions described herein allow for the production of a highly concentrated, sustained release formulation of rapamycin with enhanced drug stability and controlled release capabilities. Therapy can take the form of injectable or implantable drug depots, transdermal or mucosal patches, suppositories or coatings on medical implants as well as more traditional oral forms of administration.

The foregoing is illustrative of the present invention, and is not to be construed as limiting thereof. While embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A drug delivery composition comprising a lipophilic agent and a polymer, wherein said composition is a hydrogel, wherein the lipophilic agent exhibits sustained release, and wherein there is less than 35% lipophilic agent release within the first hour of elution.

2. A drug delivery composition comprising a lipophilic agent and a polymer, wherein said composition is a hydrogel, wherein the elution profile is substantially linear, and wherein there is less than 35% lipophilic agent release within the first hour of elution.

3. The drug delivery composition of claim 1, or claim 2, wherein the elution profile is determined through in-vitro testing.

4. The composition of claim 1 or claim 2, wherein sustained release comprises the lipophilic agent releasing over a period of >24 hours, >3 days, >1 week, >10 days, or 16 days.

5. The drug delivery composition of claim 1, or claim 2, wherein there is less than 10% agent release within the first hour of elution.

6. The drug delivery composition of claim 2, wherein the elution profile is substantially linear once a detectable amount of drug is eluted.

7. The drug delivery composition of claim 1, or claim 2, wherein the lipophilic agent comprises a limus drug, mTOR inhibitors, antibiotic agents, and/or immunosuppressive agents.

8. The drug delivery composition of claim 7, wherein the lipophilic agent comprises a limus drug selected from rapamycin, biolimus (biolimus A9), 40-O-(2-Hydroxyethyl)rapamycin (everolimus), 40-O-Benzyl-rapamycin, 40-O-(4′-Hydroxymethyl)benzyl-rapamycin, 40-O-[4′-(1,2-Dihydroxyethyl)]benzyl-rapamycin, 40-O-Allyl-rapamycin, 40-O-[3′-(2,2-Dimethyl-1,3-dioxolan-4(S)-yl)-prop-2′-en-1′-yl]-rapamycin, (2′: E,4′S)-40-O-(4′,5′-Dihydroxypent-2′-en-1′-yl)-rapamycin, 40-O-(2-Hydroxy)ethoxycar-bonylmethyl-rapamycin, 40-O-(3-Hydroxy)propyl-rapamycin, 40-O-(6-Hydroxy)hexyl-rapamycin, 40-O-[2-(2-Hydroxy)ethoxy]ethyl-rapamycin 40-O-[(3S)-2,2-Dimethyldioxolan-3-yl]methyl-rapamycin, 40-O-[(2S)-2,3-Dihydroxyprop-1-yl]-rapamycin, 40-O-(2-Acetoxy)ethyl-rapamycin 40-O-(2-Nicotinoyloxy)ethyl-rapamycin, 40-O-[2-(N-Morpholino)acetoxy]ethyl-rapamycin, 40-O-(2-N-Imidazolylacetoxy)ethyl-rapamycin, 40-O-[2-(N-Methyl-N′-piperazinyl)acetoxy]ethyl-rapamycin, 39-O-Desmethyl-39,40-O,O-ethylene-rapamycin, (26R)-26-Dihydro-40-O-(2-hydroxy)ethyl-rapamycin, 28-O-Methyl-rapamycin, 40-O-(2-Aminoethyl)-rapamycin, 40-O-(2-Acetaminoethyl)-rapamycin, 40-O-(2-Nicotinamidoethyl)-rapamycin, 40-O-(2-(N-Methyl-imidazo-2′-ylcarbethoxamido)ethyl)-rapamycin, 40-O-(2-Ethoxycarbonylaminoethyl)-rapamycin, 40-O-(2-Tolylsulfonamidoethyl)-rapamycin, 40-O-[2-(4′,5′-Dicarboethoxy-1′,2′,3′-triazol-1′-yl)-ethyl]-rapamycin, 42-Epi-(tetrazolyl)rapamycin (tacrolimus), 42-[3-hydroxy-2-(hydroxymethyl)-2-methylpropanoate]rapamycin (temsirolimus), (42S)-42-Deoxy-42-(1H-tetrazol-1-yl)-rapamycin (zotarolimus), picrolimus, novolimus, and myolimus.

9. The drug delivery composition of claim 7, wherein the lipophilic agent is rapamycin.

10. The drug delivery composition of claim 1, or claim 2, wherein the polymer comprises any one or more of a linear polymer, branched polymer, dendritic polymer, liquid crystalline polymer, amorphous polymer, semi-crystalline polymer, block co-polymer, tri-block copolymer, graft copolymer, polymer blend, ionomeric polymers, absorbable polymer, and bio-polymer.

11. The drug delivery composition of any one of claims 1-10, wherein at least a portion of the lipophilic agent is crystalline or semi-crystalline.

12. The drug delivery composition of claim 11, wherein at least 50%, 60%, 70%, 75%, 80%, 85%, 90%, 92%, 95%, 98%, 99%, 99.0%, 99.5%, or 99% of the lipophilic agent in the composition is in crystalline or semi-crystalline form.

13. The drug delivery composition of claim 1, or claim 2, formulated for use in, treatment of, or diagnosis of: chronic diseases; cancer; neurologic ailments, conditions, disorders, or diseases; pain, reproductive ailments, conditions, disorders, or diseases, or as part of a reproductive therapies or treatment; opthamalogic ailments, conditions, disorders, or diseases; GI ailments, conditions, disorders, or diseases; orthopedic ailments, conditions, disorders, or diseases; surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications.

14. The drug delivery composition of claim 1, or claim 2, wherein the composition is incorporated into a system comprising a substrate that carries the composition to the administration site or delivery site or treatment site.

15. The drug delivery composition of claim 14, wherein the substrate comprises a medical implant, diagnostic device, interventional device, and/or surgical tool.

16. The drug delivery composition of claim 14, wherein the substrate is a device, tool, or implant for use in orthopedics, neurology, cardiology, vascular treatment or diagnosis, opthamology, urology, gastroenterology, gynecology, obstetrics, aesthetic treatments or diagnosis, surgical applications such as for wound closure, healing, anti-infection, at least; dental ailments, conditions, disorders, or diseases such as periodontal disease; anti-viral treatment regimens such as HIV and hepatitis treatment or diagnosis; vaccines; and/or aesthetic applications.

17. The drug delivery composition of claim 14, wherein the substrate is a device, tool, or implant for use in allergy and immunology, anesthesiology, critical care medicine, hospice and palliative medicine, pain medicine, sleep medicine, colon and rectal surgery, dermatology, dermatopathology, emergency medicine, critical care medicine, emergency medical services, medical toxicology, sports medicine, undersea and hyperbaric medicine, family medicine, Internal medicine, cardiology, Interventional cardiology electrophysiology, endocrinology, diabetes, metabolism, gastroenterology, geriatric medicine, hematology, Infectious disease, oncology, nephrology, pulmonary disease, rheumatology, hepatology, transplant hepatology, genetics, cytogenetics, molecular genetics, neurological surgery, neurology, nuclear medicine, obstetrics, gynecology, reconstructive surgery, maternal and fetal medicine, reproductive endocrinology, endocrinology, reproductive Infertility, orthopedic surgery, orthopedics, otolaryngology, neurotology, plastic surgery, pathology, blood banking/transfusion medicine, cytopathology, dermatopathology, neuropathology, pediatrics, neonatal-Perinatal medicine, neurodevelopmental disabilities, hematology, pulmology, rheumatology, aerospace medicine, occupational medicine, psychiatry, brain injury medicine, neurophysiology, epilepsy, neuromuscular medicine, neuroradiology, nuclear radiology, radiation oncology, medical physics, vascular and Interventional radiology, surgery, vascular surgery, thoracic and cardiac surgery, and urology.
